(19) United States
(12) Patent Application Publication
(10) Pub. No.: US 2020/0360569 A1
(43) Pub. Date: Nov. 19, 2020

(54) POLYMERIC STRUCTURES

(71) Applicant: Ecole Polytechnique Federale De Lausanne, Lausanne (CH)

(72) Inventors: Esther Amstad, Lausanne (CH); Huachuan Du, Lausanne (CH)

(21) Appl. No.: 16/809,079

(22) Filed: Mar. 4, 2020

Related U.S. Application Data
(63) Continuation of application No. PCT/EP2018/074059, filed on Sep. 6, 2018.

(30) Foreign Application Priority Data
Sep. 6, 2017 (GB) 1714282.9

(51) Int. Cl. A61L 27/56; A61L 27/52; C08J 3/075; C08J 3/24

(52) U.S. Cl. CPC A61L 27/56; A61L 27/52; C08J 2333/14; C08J 3/245; C08J 3/075

(57) ABSTRACT

A polymeric structure comprising a plurality of spherical or polyhedral polymeric cells bound through their sidewalls via a connection element, wherein the polymeric cells and the connection element have a different Young's modulus.

### TECHNICAL FIELD

The invention lies in the field of material science. More particularly, the invention relates to mechanically heterogeneous polymeric structures and methods for producing thereof.

### BACKGROUND ART

Natural soft materials are often composed of different types of proteins that self-assemble into hierarchical structures. For example, the cytoskeleton is made of proteins and a large fraction of them assembles into filaments. These proteins are essential for the cell because they help maintain its structure and organization. Moreover, they are crucial for cell movement and division.

Similarly, the mussel byssus that allows mussels to strongly adhere to solid surfaces such as rocks and ships is made of different proteins that are assembled into a hierarchical structure. Many of the natural soft materials display a remarkable combination of strength and toughness. For example, the Young's modulus of the mussel byssus of mytilus edulis or that of Mytilus galloprovincialis can reach values up to 500 MPa whereas its ultimate tensile stress reaches values up to about 160 MPa.

Inspired by the excellent mechanical properties of these natural soft materials, a lot of research is devoted towards the design of hydrogels that display similar mechanical properties.

Despite this research, there remains a need for polymeric structures with improved mechanical properties.

### SUMMARY OF THE INVENTION

In a first aspect of the present invention there is provided a polymeric structure, wherein the polymeric structure comprises a plurality of spherical or polyhedral polymeric cells bound through their sidewalls via a connection element, wherein the polymeric cells and the connection element have a different Young's modulus.

The polymeric structures of the present invention mirror the mechanical behaviour of natural polymers.

In a further aspect of the present invention there is provided a polymeric structure, wherein the polymeric structure comprises a plurality of spherical or polyhedral polymeric cells bound through their sidewalls via a connection element, wherein the polymeric cells and the connection element have different crosslink densities.

Different crosslink densities mimic natural polymers in terms of arrangement of the chemical bonds among those precursors. There are various ways in which different crosslink densities can be achieved. For example, tuning polymerisation kinetics can control the relative crosslink densities within and between the cells. Surface modifying agents can also be used to achieve different crosslink densities. Also, selecting materials with different properties for the polymeric cells and connection element can give different crosslink densities.

The polymeric structures of the present invention are intended to mimic the structure of some natural polymers and may overcome limitations of the prior approaches concerning the production of polymeric structures, such as hydrogel scaffolds.

The polymeric structures of the present invention may be used to produce biocompatible polymeric structures sufficiently mechanically stable to be suitable for tissue engineering application, as e.g. bioscaffolds or tissue substitution.

The polymeric structures of the present invention may enable the creation of a polymeric platform whose features in terms of, e.g., size, shape, mechanics or optics can be locally tuned at a very high resolution.

In an embodiment, the connection element is a polymeric connection element.

In an embodiment, the polymeric cells and the polymeric connection element are composed of the same polymeric matrix.

In a further embodiment, the polymeric cells and the polymeric connection element are composed of a different polymeric matrix.

In a yet further embodiment, the polymeric connection element comprises crosslinked surface modifying agents.

In an embodiment, the Young's modulus of the polymeric cells is higher compared to the Young's modulus of the connection element. The Young's modulus between adjacent cells is lower than the Young's modulus of the cells. In an alternative embodiment, the Young's modulus between adjacent cells is higher than the Young's modulus of the cells.

In an embodiment, the crosslink density of the polymeric cells is different compared to the crosslink density of the connecting element. In a preferred embodiment, the crosslink density of the polymeric cells is higher compared to the crosslink density of the connection element. The crosslink density between adjacent cells is lower than the crosslink density within cells. Alternatively, the crosslink density between adjacent cells is higher than the crosslink density within cells.

In an embodiment, at least part of the polymeric cells are adjacently disposed in a crystalline lattice along or within the structure.

In an embodiment, the polymeric structure is shaped as a flat sheet or a three-dimensional solid.

In an embodiment, the polymeric structure is a flat sheet comprising a non-flat surface.

In an embodiment, at least part of the polymeric cells are labelled with a marker or a dye, such as a fluorescent dye, crystal label, electronic marker and/or a dye that is visible to the human eye.

In a further embodiment, the grain boundaries may be functionalised. The functionalisation may be an electrically conducting material or a magnetic material. The electrically conducting material may be metal nanoparticles (for example aluminium nanoparticles) or may be graphene. The magnetic material may be based on iron, such as iron oxide nanoparticles or may be on nickel-based materials.

In a particular embodiment, the present invention comprises macroscopic structured hydrogel sheets. The sheets may be made of regularly arranged, covalently crosslinked hexagonal prismatic hydrogel microparticles. The sheets may display a narrow size distribution, preferably of 10-200 μm diameter.

The structure, local composition, and Young's modulus of the polymeric structures of the present invention can be tuned with the size and composition of microparticles. The shape and morphology of these structures can be adjusted with the polymerization kinetics. Moreover, these structures display a heterogeneous crosslink density. The uneven crosslink density increases the Young's modulus of the structured sheets or three-dimensional solids of the present invention compared to their unstructured counterparts, thereby opening new possibilities to tune the structure and mechanical properties of the materials of the present invention.

In a yet further embodiment, the surface roughness of the polymeric structure can be modified. In an embodiment, the polymeric cells may comprise a semi-spherical surface. The semi-spherical surface may be a dome. The size of the dome may be tuned depending on the desired degree of surface modification.

In a yet further embodiment, the polymeric structure is self-healing.

In a further aspect of the present invention there is provided a method for producing a polymeric structure comprising a plurality of spherical or polyhedral polymeric cells bound through their sidewalls via a connection element, wherein the polymeric cells and the connection element have a different Young's modulus, said method comprising the steps of:
a) providing a plurality of spherical or polyhedral polymeric cells disposed on a support substrate;
b) providing a connection element between the sidewalls of the polymeric cells; and
c) allowing a crosslinking reaction between the polymeric cells and the connection element by providing a crosslinking trigger.

In a yet further aspect of the present invention there is provided a method for producing a polymeric structure comprising a plurality of spherical or polyhedral polymeric cells bound through their sidewalls via a connection element, wherein the polymeric cells and the connection element have non-homogeneous crosslink densities, said method comprising the steps of:
a) providing a plurality of spherical or polyhedral polymeric cells disposed on a support substrate;
b) providing a connection element between the sidewalls of the polymeric cells; and
c) allowing a crosslinking reaction between the polymeric cells and the connection element by providing a crosslinking trigger.

The mechanical properties of the polymeric structure can be tuned by controlling the crosslink densities during the polymerization process.

In an embodiment, the step of providing a plurality of spherical or polyhedral polymeric cells disposed on support substrate in a packed arrangement comprises:
a) providing a plurality of drops comprising chemical precursors of a polymeric matrix composing the polymeric cells, said drops being arranged into a non-chemically bound packed arrangement on said support substrate; and
b) starting a polymerization reaction of the polymeric matrix composing the polymeric cells.

The support substrate may be a solid support substrate. Alternatively, the support substrate may be formed from, e.g. a liquid/liquid interface or a liquid/air interface.

In an embodiment, the plurality of drops comprising chemical precursors of a polymeric matrix are in a first phase and provided as an emulsion in an immiscible second phase.

A crosslinking trigger is provided to start the polymerisation reaction of the precursor monomers. During the polymerization reaction, but before the polymerization reaction is complete, the second phase is removed.

Because the emulsion drops display a narrow size distribution (e.g. the coefficient of variation, defined as the standard deviation of the drop diameter divided by its mean, is below 7%), they self-assemble into a close-packed structure. The crosslinking trigger starts the formation of the spherical or polyhedral polymeric cells by causing monomers to crosslink. As the second phase is removed the cells start to deform and become non-spherical. As the second phase continues to be removed the cells rupture. Because the polymerization reaction is incomplete at the time of the cell rupture, the unreacted monomers that are located at the surface of the cells crosslink adjacent cells. Through this process, the connection element is formed. Thus, the polymeric cells and connection element are formed from the same chemical precursor but have different properties because the initial cell polymerisation to form the polymeric cells; subsequent rupture due to second phase evaporation; and secondary crosslinking of unreacted monomers to form the connection element give rise to different crosslink densities and Young's modulus for the polymeric cell and connection element. By controlling the polymerisation rate and the second phase evaporation rate it is possible to control the crosslink densities of both the polymeric cell and connection element, which allows the mechanical properties of the polymeric structure to be tuned.

In an embodiment of the present invention there is provided a method for producing a polymeric structure comprising a plurality of spherical or polyhedral polymeric cells bound through their sidewalls via a connection element, wherein the polymeric cells and the connection element have different crosslink densities and/or Young's modulus, said method comprising the steps of:
a) providing an emulsion comprising:
   a first phase comprising a plurality of drops of chemical precursors of a polymeric matrix dispersed into an immiscible second phase; and
   a crosslinker;
b) disposing said emulsion on a support substrate so that the drops assemble into a non-chemically bound packed arrangement;
c) starting a polymerization reaction of the polymeric matrix by providing a crosslinking trigger; and
d) during the polymerization reaction, and before the polymerization reaction is complete, allowing the removal of the second phase.

In an embodiment of the method, the first phase is an aqueous phase and the second phase is an organic phase.

In an embodiment of the method, the crosslinker is a portion of the chemical precursors' molecules.

In an embodiment of the method, the chemical precursors comprise a minimum monomeric concentration of greater than 30 wt %, greater than 40 wt %, greater than 50 wt %, or greater than 60 wt %.

In a further embodiment of the present invention, surface modifying agents can be used to achieve crosslinking between the polymeric cells. The use of surface modifying agents to form the connection element enables tuning of the properties of the polymeric structure by enabling different Young's modulus and/or crosslink densities between the cells and the connection element. In this embodiment, the cells may or may not rupture before the polymerisation reaction is complete.

Thus in an embodiment of the present invention, the connection element is formed using a surface modifying agent. In this embodiment, surface modifying agent(s) provide the connection element between the sidewalls of the polymeric cells.

In a yet further embodiment of the present invention, the polymerisation is allowed to continue to completion before cell rupture. This provides a plurality of spherical or polyhedral polymeric cells which are not connected together. In this embodiment, a second chemical precursor of a polymeric matrix (either the same or different precursors to that used to form the polymeric cells) can be added to form the connection element. This second precursor is then crosslinked to connect the polymeric cells into a polymeric structure. The reaction conditions including the choice of crosslink density and/or choice of chemical precursor(s) can be tuned to achieve different Young's modulus and/or crosslink densities between the polymeric cells and connection element. In this embodiment, polymeric cells are first produced and then a second polymer is added to backfill and create the connection element.

Thus, in an embodiment of the present invention, the step of providing a connection element between the sidewalls of the polymeric cells comprises:
a) disposing a liquid or semi-solid connection element; and
b) optionally allowing solidification or polymerization of said liquid or semi-solid connection element.

There are therefore various ways in which polymeric structures of the present invention can be formed.

In some embodiments, the polymeric cells and connection element are formed from the same chemical precursors, but by tuning the polymerisation kinetics, different crosslink densities and/or Young's modulus are obtained. An example of this is by controlling the polymerisation kinetics and second phase evaporation such that the polymeric cells form but rupture before polymerisation is complete. This allows unreacted monomers to migrate to the surface of the ruptured cells and subsequently crosslink to adjacent cells creating the connection element.

In further embodiments, surface modifying agent(s) is/are used to facilitate formation of the connection element. In these embodiments, the polymeric cells may undergo rupture as described above or alternatively may not undergo rupture before the cell polymerisation is complete. Again, by tuning the surface modifying agents, different Young's modulus and/or crosslink densities can be obtained.

In yet further embodiments, the polymeric cells can be allowed to form and then a separate connection element is added to connect the cells and form the polymeric structure. These backfill embodiments allow tuning of the properties of the polymeric cells vs connection element by selection of precursors and/or reaction conditions.

Any suitable crosslinking trigger may be used with the embodiments of the present invention. In an embodiment, the crosslinking trigger is a thermal trigger. In a further embodiment, the crosslinking trigger is a light trigger. In a particular embodiment, the crosslinking trigger is a UV trigger. Crosslinkers can also be ions, nanoparticles, catalysts, chemically reactive organic compounds, a chemical trigger such as a complexation agent, or a catalyst. Any suitable crosslinking trigger appropriate for these crosslinkers may be used.

Where the embodiments of the present invention involve removal of a second phase, the crosslinking trigger may also cause removal of said second phase.

In an embodiment of the present invention, the method further comprises a photoinitiator.

In an embodiment of the present invention, the step of providing an emulsion is performed through a microfluidic device.

In an embodiment, the method further comprises a step of labelling at least part of the plurality of polymeric cell or drops with a marker or a dye, such as a fluorescent dye crystal label, electronic marker and/or a dye that is visible to the human eye.

In an aspect of the present invention there is provided a pharmaceutical composition comprising the above-described polymeric structures.

In a further aspect of the present invention there is provided the use of the polymeric structure of the invention as a scaffold for tissue engineering.

In a yet further aspect of the present invention there is provided a physical, non-printed polymeric data matrix tag, wherein it comprises a polymeric structure shaped as a flat sheet comprising a non-flat surface, and/or in which part of the polyhedral polymeric cells are labelled with a marker or a dye, such as a fluorescent dye, crystal label, electronic marker and/or a dye that is visible to the human eye.

### BRIEF DESCRIPTION OF THE DRAWINGS

In the Figures:

FIG. 1A shows an optical micrograph of a section of a millipede microfluidic device in operation. The arrows indicate the flow direction of the inner aqueous solution, and the white arrows indicate the flow direction of the HFE 7500 oil.

FIG. 1B is an optical micrographs of water in oil emulsion drops produced in 40 μm tall nozzles that are illuminated with UV light to convert them into hydrogel microparticles. The microparticles display a coefficient of variation below 2.5%.

FIG. 1C shows these hydrogel particles dispersed in water.

FIG. 2A shows an optical micrograph of hydrogel particles assembled into a hexagonal close packed structure.

FIG. 2B shows an optical micrograph of hydrogel particles after water evaporation, where the hexagonal structure becomes defective because adjacent particles are not cross-linked.

FIG. 3 depicts a schematic illustration of the formation of structured hydrogel sheets. Emulsion drops are deposited on a substrate where drops arrange into the energetically most favorable hexagonally close packed structure. Once the majority of the oil is evaporated, drops deform and attain a hexagonal prismatic shape. They are subsequently illuminated with UV light to convert them into hydrogel particles and to covalently link adjacent particles.

FIGS. 4A-4C show time-lapse optical micrographs of self-assembled drops taken during the evaporation of the surrounding oil. FIG. 4A shows monodisperse drops self-assemble into a hexagonal close packed structure. FIG. 4B shows the drops deform while the oil evaporates. FIG. 4C shows the drops eventually attain a hexagonal prismatic shape.

FIG. 4D shows a SEM micrograph of individual hexagonal prismatic particles, obtained by polymerizing the monomers before drops ruptured.

FIGS. 4E-4F show (FIG. 4E) top view and (FIG. 4F) side view SEM micrographs of structured hydrogel sheets made from drops that were self-assembled on a hydrophilic glass slide.

FIG. 5A shows an optical micrograph of a hydrogel sheet made by self-assembling drops on a PDMS substrate and subsequently illuminating them with UV light to convert them into hydrogels. The entire sample is shown in the inset. FIG. 5B top view and FIG. 5C side view shows SEM micrographs of a structured hydrogel sheet made from microparticles that are covalently linked to each other. The sample is cut to visualize the connection between adjacent particles.

FIG. 6 shows stress vs. strain curves of unstructured (squares) and structured (circles) PEG700-DA hydrogel sheets. Optical micrographs of the corresponding ruptured sheets are shown in the insets.

FIG. 7A shows a SEM micrograph of a hydrogel sheet composed of spherical particles arranged in a cubic lattice. The surfaces of drops represent the structure of the hexagonal wells used to assemble drops into a cubic lattice.

FIG. 7B shows a SEM micrograph of a structured hydrogel sheet composed of hexagonal prismatic particles each one containing semi-spherical domes with a side view of a single particle in the inset.

FIGS. 7C-7E show optical micrographs of self-assembled drops containing hydrogel particles made by illuminating self-assembled PEG-DA containing drops with a UV light source located (FIG. 7C) 15 cm, (FIG. 7D) 10 cm and (FIG. 7E) 4 cm away from the sample surface.

FIGS. 8A-8D show fluorescent micrographs of hydrogel sheets composed of a mixture of FITC-dextran labeled and non-labeled microparticles with a diameter of (FIG. 8A) 120 μm, (FIG. 8C) 67 μm, and (FIG. 8D) 36 μm. FIG. 8B shows the normalized fluorescent intensity profile measured along the line in FIG. 8A.

FIG. 9 shows an optical micrograph of a bilayer of hexagonal prismatic particles after they have been mechanically stressed.

FIG. 10 shows an optical micrograph of a granular hydrogel composed of polydisperse cells.

FIG. 11 shows a scanning electron micrograph of a multilayer of polygonal cells that are covalently linked to each other.

### DESCRIPTION OF EMBODIMENTS

The present disclosure may be more readily understood by reference to the following detailed description presented in connection with the accompanying figures, which form a part of this disclosure. It is to be understood that this disclosure is not limited to the specific conditions or parameters described and/or shown herein, and that the terminology used herein is for the purpose of describing particular embodiments by way of example only and is not intended to be limiting of the claimed disclosure.

As used herein and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where descriptions of various embodiments use the term “comprising”, those skilled in the art would understand that in some specific instances, an embodiment can be alternatively described using language “consisting essentially of” or “consisting of.”

The invention is directed to a new kind of polymeric structure and associated methods for its production, wherein the structure, shape, and surface roughness of these structures can be tuned with the size and arrangement of the blocks or cells composing it. Moreover, the method developed to produce those structures allows for easy regulation of the polymerization kinetics of the precursors of the polymeric matrix composing the material of the invention, opening up new possibilities to tune the mechanical properties thereof by controlling the crosslink densities during the polymerization process. The so-obtained structured polymeric matrix have the potential to allow the design of biomimetic soft materials whose properties more closely resemble those of natural materials, thus paving the way to e.g. use them for tissue engineering or screening purposes.

The polymeric structure of the invention is characterized in that it comprises a plurality of spherical or polyhedral polymeric cells. In the frame of the present disclosure, a “cell” is the single building block composing the polymeric matrix of the structure of the invention. The polymeric cells of the present invention may also herein be described as cells, particles, polymeric particles, microparticles or hydrogel microparticles. A “polyhedron” is a solid in three dimensions with flat polygonal faces, straight edges and sharp corners or vertices. Another common definition given for a polyhedron is that of a solid whose boundary can be covered by finitely many planes, or that it is a solid formed as the union of finitely many convex polyhedra. Polyhedra may be classified and are often named according to the number of faces; the naming system is based on Classical Greek, for example tetrahedron (4), pentahedron (5), hexahedron (6), triacontahedron (30) and so on. Depending on the circumstances, the polyhedral cells of the polymeric structure of the invention could have slightly curved polygonal faces, and/or rounded edges and/or corners. Accordingly, the polyhedral cells of the invention could be almost polyhedral in nature, and it will be apparent for a person skilled in the art that the polyhedron definition given above must not be interpreted in a limiting sense.

In one embodiment, the polyhedral cell according to the above definition is a prismatic cell. A “prism” is a polyhedron comprising an n-sided polygonal base, a second base which is a translated copy of the first, and n other faces (necessarily all parallelograms) joining corresponding sides of the two bases. All cross-sections parallel to the bases are translations of the bases. Prisms are named for their bases, so a prism with a pentagonal base is called a pentagonal prism. In one embodiment according to the invention, the prismatic cell is a hexagonal prismatic cell.

The polymeric cells can have dimensions (e.g., diameter, side or diagonals, depending on the shape of the cells) in the order of nanometres up to millimetres or even centimetres; for example, a single polymeric cell can have a diameter, side or diagonal spanning from 10 nm to 10mm, such as for instance from 100 nm to 10 mm, from 100 nm to 1 mm, from 100 nm to 500 μm, from 100 nm to 100 μm, from 500 nm to 300 μm, from 1 μm to 200 μm, from 10 μm to 200 μm, from 20 μm to 180 μm, from 30 μm to 160 μm, from 40 μm to 140 μm, from 40 μm to 120 μm and so forth. Preferably the range is the cell diagonal. Particularly preferred is between 1 μm and 200 μm. Moreover, the plurality of polymeric cells within the polymeric structure of the invention can be composed of a mix of cells of different sizes and/or shapes.

It is to be understood that the polymeric cells of the invention can be fully or partly composed of a polymeric matrix. For instance, microcapsules (also known as core-shell capsules), comprising their core or their shell in a polymeric form, can be envisaged as building blocks for composing the polymeric structure according to the invention. In this embodiment, advantageous additional properties can be implemented within the polymeric structure; for instance, a polymeric shell of a microcapsule could set the physical boundary of a tiny microreactor represented by the core of the capsule, which could comprise chemical or biological matter for e.g. driving cell differentiation.

Within a polymeric structure of the invention, the cells are bound through their sidewalls via a connection element, wherein the polymeric cells and the connection element have a different Young's modulus. The cells can be bound chemically, ionically, physically (including with covalent and/or ionic bonds; this definition does not include weak non-covalent interactions such as Van der Waals interactions).

The connection element can in some embodiments comprise or substantially consist of a polymeric material, and the polymeric cells and the polymeric connection element can be composed of the same or different polymeric matrices. As used herein, a “polymeric matrix” is meant to be a material comprising polymers. The wording “polymeric material” will be used herein interchangeably to refer to said polymeric matrix, wherever needed.

The connection element may encompass a single connected matrix or may comprise multiple elements. The connection element(s) connects the cells.

A “polymeric material” is any material comprising polymers, large molecules (also known as macromolecules) composed of many repeated smaller units, or subunits, called monomers, tightly bonded together by ionic or covalent bonds. Polymer architecture at the molecular scale can be rather diverse. A linear polymer consists of a long linear chain of monomers. A branched polymer comprises a long backbone chain with several short side-chain branches covalently attached. Cross-linked polymers have monomers of one long or short chain covalently bonded with monomers of another short or long chain. Cross-linking results in a three-dimensional molecular network; the whole polymer is a giant macromolecule. Another useful classification of polymers is based on the chemical type of the monomers: homopolymers consist of monomers of the same type, copolymers have different repeating units. Furthermore, depending on the arrangement of the types of monomers in the polymer chain, there are the following classification: the different repeating units are distributed randomly (random copolymer) or there are alternating sequences of the different monomers (alternating copolymers) in block copolymers long sequences of one monomer type are followed by long sequences of another type; and graft copolymers consist of a chain made from one type of monomer with branches of another type. A sufficiently dense polymer solution can be crosslinked to form a polymer gel, including a hydrogel or a cryogel, which is a soft solid.

Polymer materials may also be formed by blending two or more polymers into physical mixtures. For example, the rather poor impact strength of polystyrene is greatly improved by incorporating small particles of an elastomer. Many properties of polymeric materials depend on the microscopic arrangement of their molecules. Polymers can have an amorphous (disordered) or semicrystalline (partially crystalline, partially ordered) structure. Polymers can be mixed with inorganic particles (usually in the form of continuous fibres, such as glass or particulates such as mica, talc and clay) in order to modify and improve (mainly but not exclusively) their mechanical properties.

The polymeric matrix according to the invention may comprise one or more compounds selected from a non-exhaustive list comprising natural polymeric material (i.e., non-synthetic polymers, polymers that can be found in nature) and/or polymers derived from Extra Cellular Matrix (ECM) as gelatin, elastin, collagen, silk, agar/agarose, chitosan, fibrin, proteoglycans, a polyamino-acid or its derivatives, preferably polylysin or gelatin methyl cellulose, carbomethyl cellulose, polysaccharides and their derivatives, preferably glycosaminoglycanes such as hyaluronic acid, chondroitinsulfate, dermatansulfate, heparansulfate, heparine, keratansulfate or alginate, as well as any derivative thereof, fragment thereof and any combination thereof. Particularly preferred compounds include polyamino-acids and polysaccharides. Especially preferred compounds are polysaccharides.

Each monomer may have at least three reactive sites.

A thermoset material can also be envisaged for use according to the present disclosure. A “thermoset” is a pre-polymer in a soft solid or viscous state that changes irreversibly into an infusible, insoluble polymer network by curing. Curing is induced by the action of heat or suitable radiation, often under high pressure. The curing process transforms the resin into a plastic or rubber by cross-linking individual chains of the polymer. The cross-linking is facilitated by energy and catalysts at chemically active sites, which may be unsaturated sites or epoxy sites, for example, linking into a rigid, three-dimensional structure. This yields molecules with a large molecular weight, resulting in a material that usually decomposes before melting. Therefore, a thermoset cannot be melted and re-shaped after it is cured.

Examples of thermoset materials include, but are not limited to, alkyds, epoxies, phenolics (e.g., Bakelite), polyimides, formaldehyde resins (e.g., urea formaldehyde or melamine formaldehyde), polyester thermosets, unsaturated polyesters, polyurethane, bis-maleimides (BMI), silicone materials such as polydimethylsiloxane (PDMS) and any combination thereof.

Further suitable materials according to the present invention may comprise one or more compounds selected from a non-exhaustive list comprising poly(lactic-co-glycolic acid), lactide and glycolide polymers, caprolactone polymers, hydroxybutyric acid, polyanhydrides, polyesters, polyphosphazenes, polyphosphoesters and poly(glycerol sebacate acrylate), polypropylene, polypropylenoxide or their derivatives, polymethylenoxide or its derivatives, polyethylene or its derivatives such as polyethylene glycole (PEG), polyethylenoxide or their derivatives, polyacrylate or its derivatives, poly(vinyl alcohol) (PVA) and copolymers, poly(vinylpyrrolidone) (PVP), Poly(N-isopropylacrylamide) (PNIPAM), Po/y(acrylic acid) (PAA) and combinations thereof.

For the sake of clarity, Young's modulus, also known as the elastic modulus, is a measure of the stiffness of a solid material. It is a number that measures an object or substance's resistance to being deformed elastically (i.e., non-permanently) when a force is applied to it. Young's modulus is a mechanical property of solid materials which defines the relationship between stress (force per unit area) and strain (proportional deformation) in a material. The elastic modulus of an object is defined as the slope of its stress-strain curve in the elastic deformation region: a stiffer material will have a higher elastic modulus. Depending on the needs and circumstances, the Young's modulus of the polymeric cells can be higher or lower compared to the Young's modulus of the connection element. Said Young's modulus can span for instance from 1 kPa and about 100 MPa, such as for instance between 10 and 100 kPa, between 10 kPa and 10 MPa, between 10 and 100 MPa, between 1 and 10 MPa, about 1 kPa to about 1000 kPa, about 1 kPa to about 100 kPa or about 1 kPa to about 10 kPa.

The polymeric structures according to the present invention may be self-healing.

A “self-healing” polymeric structure means the structure is able to spontaneously repair if it is breached. The structure is able to reform the bond between cells and connection element, reform the cells themselves and/or reform the connection element, to reform an intact structure. The repair may reform the structure to its original structure or it may reform the structure in a slightly different structure that nevertheless repairs the breach.

Any suitable method to form a self-healing polymeric structure is encompassed by the present invention. An example of a suitable method is by using metal-coordinating group or groups reversibly cross-linked with suitable cations to achieve self-healing properties.

“Metal coordinating group” means a group which is able to coordinate with a metal cation by forming a reversible ionic bond between the coordinating group and the metal cation.

The polymers of the present invention can optionally be functionalized with metal coordinating groups. These groups may then crosslink with the metal cation to form reversible bonds that impart self-healing properties to the materials.

The ratio of metal coordinating group(s) to metal ions can be tuned. There may be one, two or three coordinating groups per metal ion.

Preferred metal coordinating groups are benzenediol or derivatives thereof. Further preferred metal coordinating groups are benzenetriol or derivatives thereof. Further metal coordinating groups might be histidines or derivatives thereof, groups comprising a carboxyl group; and ethylenediaminetetraacetic acid and derivatives thereof. Preferred metal coordinating groups are benzenediol or benzenetriol. Particularly preferred metal coordinating groups are benzenediol or derivatives thereof.

“Benzenediol” means a benzene ring substituted with two hydroxyl groups and “Benzenetriol” means a benzene ring substituted with three hydroxyl groups. The benzene ring may optionally be further substituted. To provide sufficient complexation, the hydroxyl groups are adjacent to each other, e.g., in a benzenediol the ortho (catechol) isomer. Thus, in a preferred embodiment, the metal coordinating group is catechol (also known as 1,2-benzenediol) or a derivate thereof. For a benzenetriol, a preferred molecule is gallol.

In a preferred embodiment, two hydroxyl groups are in the ortho-meta positions. In an alternative embodiment, two catechol hydroxyl groups are in the meta-para positions. The meta-para position is especially preferred.

Further metal coordinating groups include specific catechols (such as dopamine, hydrocaffeic acid, and tiron (disodium 4,5-dihydroxy-1,3-benzenedisulfonate).

Yet further metal coordinating groups include amino acids. Suitable amino acids include histidine, serine, threonine, asparagine, glutamine, lysine, or cysteine.

“Metal cation” can be any metal cation suitable to coordinate with a metal coordinating group. The metal cation forms reversible ionic bonds with metal coordinating group(s). Suitable metal cations include metal ions, metal oxides, metal hydroxides, metal carbides, metal nitrides and/or metal nanoparticles.

Particular metal ions include beryllium, magnesium, calcium, strontium, barium, chromium, manganese, iron, cobalt, nickel, copper, silver, gold, zinc, cadmium, mercury, aluminium, gallium, indium, tin, lead and bismuth. Particularly preferred metal cations include iron, aluminium or titanium, with iron especially preferred.

More particularly, suitable cations include Be²⁺ beryllium ion, Mg²⁺ magnesium ion, Ca²⁺ calcium ion, Sr²⁺ strontium ion, Ba²⁺ barium ion, Ti²⁺ titanium (II), Ti⁴⁺ titanium (IV), Cr²⁺ chromium (II), Cr³⁺ chromium (III), Cr⁶⁺ chromium (VI), Mn²⁺ manganese (II), Mn³⁺ manganese (III), Mn⁴⁺ manganese (IV), Fe²⁺ iron (II), Fe³⁺ iron (III), Co²⁺ cobalt (II), Co³⁺ cobalt (III), Ni²⁺ nickel (II), Ni³⁺ nickel (III), Cu⁺ copper (I), Cu²⁺ copper (II), Ag⁺ silver ion, Au⁺ gold (I), Au⁺³ gold (III), Zn²⁺ zinc ion, Cd²⁺ cadmium ion, Hg₂²⁺ mercury (I), Hg²⁺ mercury (II), Al³⁺ aluminium ion, Ga³⁺ gallium ion, In⁺ indium (I), In³⁺ indium (III), Sn²⁺ tin (II), Sn⁴⁺ tin (IV), Pb²⁺ lead (II), Pb⁴⁺ lead (IV), Bi³⁺ bismuth (III), and Bi⁵⁺ bismuth (V). Particularly preferred metal cations include Fe³⁺ iron (III).

The metal may be added in the form of a metal salt. Suitable metal salts include but are not limited to halides, nitriles, hydroxides and the like.

The metal cation may be in the form of an oxide or nanoparticle. For example, iron oxide nanoparticles may be used. Other suitable oxides or nanoparticles include iron oxides, iron nitrides, iron carbides, nickel oxides, nickel carbides, titanium oxides, titanium metal particles, titanium nitrides, titanium carbides.

Using nanoparticles allows for larger numbers of metal coordinating groups to ionically bond with a single nanoparticle, which may impact the properties of the material.

“Metal complexed” or “metal coordinated” means coordination via their metal coordinating groups and metal cations through complexation. Through this coordination, the material can form reversibly cross-linked structures that can self-heal via further reversible cross-linking.

The polymeric structure of the invention can be in many advantageous embodiments a soft structure. In the frame of the present disclosure, a “soft” material or structure is any material or structure that is either compressible, flexible, elastic, has memory shape properties or any combination thereof. If intended to be used in living subjects, moreover, the material may be a biocompatible and/or sterilisable material suitable for medical uses. Advantageously, a soft polymeric structure according to some embodiments of the invention can be produced as a polymeric gel structure, such as a polymeric hydrogel.

As used herein, the term “gel” refers to a non-fluid colloidal network or polymer network that is expanded throughout its whole volume by a fluid. A gel is a solid three-dimensional network that spans the volume of a liquid medium and ensnares it through surface tension effects. The internal network structure may result from physical bonds (physical gels) or chemical bonds (chemical gels).

As used herein, the term “hydrogel” refers to a gel in which the swelling agent is water. A hydrogel is a macromolecular polymer gel constructed of a network of crosslinked polymer chains. It is synthesized from hydrophilic monomers, sometimes found as a colloidal gel in which water is the dispersion medium. Hydrogels are highly absorbent (they can contain over 90% water) natural or synthetic polymeric networks. As a result of their characteristics, hydrogels develop typical firm yet elastic mechanical properties.

Several physical properties of the (hydro)gels are dependent upon concentration.

Increase in (hydro)gel concentration may change its pore radius, morphology, or its permeability to different molecular weight proteins. One skilled in the art will appreciate that the volume or dimensions (length, width, and thickness) of a (hydro)gel can be selected based on instant needs, such as for instance the region or environment into which the (hydro)gel is to be implanted if used in a biomedical setting.

The polymeric structures of the present invention may be formed on a support substrate. The substrate may be a solid support. Alternatively, it may be formed via a liquid/liquid interface or a liquid/air interface. The support substrate enables the polymeric cells to form into a packed arrangement so that the polymeric structure forms.

The polymeric structure of the invention can be in the form of three-dimensional solid or as a flat sheet. As a way of example, a three-dimensional solid can be any kind of polyhedral such as a cube, parallelogram, pyramid, tetrahedron and so on, or customized shapes can be imagined such as e.g. conic or spherical structures. In some embodiments however, a flat sheet is provided, said sheet being a thin structure in which the thickness is much smaller, such as at least one order of magnitude, compared to the other dimensions. In non-limiting embodiments, the thickness of this sheet or layer of the polymeric structure can be comprised between 1 μm and 1 cm,1 μm and 1 mm, such as for instance between 10 μm and 100 μm. A polymeric structure in the form of a flat sheet can also comprise one non-flat surface, such as for instance an upper and/or bottom surface having a buckled or wavy profile. In this embodiment, the non-flat profile of the sheet surface can be provided by a dome-like shape of the surface of one or more polyhedral cells.

It is possible to build polymeric structures comprising multiple layers of cells. The structure may comprise, for example, two layers of cells, three layers, five or more layers, ten or more layers and the like.

The polymeric structure of the invention can show an amorphous, semicrystalline or crystalline topology. In particular embodiments of the invention, however, the two- or even three-dimensional shape of the polymeric structures has a crystal architecture based on a crystalline lattice. In this preferred embodiment of the invention, the polymeric cells, such as polyhedral polymeric cells, comprised in the polymeric structure are adjacently disposed along or within the material as regularly tessellated units repeated in two or three dimensions. When referring to a flat sheet structure, the lattice can be composed of one or more different polygonal or quasi-polygonal shapes, such as square, pentagons, hexagons and so on. In one embodiment, the crystalline structure is made up from regularly tessellated hexagonal units repeated in two dimensions, which result in a planar honeycomb two-dimensional crystal lattice. When referring to a three dimensional polymeric structure, the lattice can be composed of one or more different polyhedra, such as pyramids, tetrahedron, pentahedron, hexahedron and the like.

The polymeric structure of the invention can be used for instance as a scaffold for biomedical applications (as well as part of a scaffold), such as e.g. tissue engineering, tissue reconstruction, as a platform for in vitro or ex vivo cell culture and so forth. Nature produces soft materials with unprecedented mechanical properties. These materials contain a mixture of covalent and ionic bonds that are spatially well-separated. At present, the distribution of the crosslinks during the polymeric structures' manufacturing is not controlled, and therefore the local softness/stiffness cannot be controlled with high accuracy. The polymeric structure of the present invention offers the possibility to generate soft materials with a well-defined heterogeneous distribution of ionic and covalent bonds. This may enable significantly better mechanical properties than is currently achievable, particularly for the design of tough and strong soft materials such as artificial tendons, ligaments, meniscus and the like.

Moreover, since the polymeric structure of the present invention offers means to spatially vary the composition and mechanical properties of hydrogels (and other gels), this could offer new possibilities in tissue engineering because different cell types require different mechanical properties of the scaffolds. Furthermore, even the same stem cell type differentiates differently if contained in matrices with different stiffness. The currently developed material might enable to design 2D or even 3D scaffolds whose mechanical properties can be adjusted such that tissues composed of different cell types whose location is well defined can be fabricated. This might open up new possibilities directed towards the goal of growing functional artificial organs.

In the frame of the present disclosure, a “scaffold” is any three dimensional structure having a framework architecture, i.e. a support structure comprising hollow spaces within it. Generally speaking, a scaffold material is an artificial structure capable of supporting three-dimensional body tissue/organ formation in vivo, ex vivo or in vitro. In this context, a scaffold material is also referred herewith as a “biomaterial” or “bioscaffold”. A bioscaffold, inter alia, allows cell attachment and migration, delivers and retains cells and biochemical factors, enables diffusion of vital cell nutrients and expressed products, exerts certain mechanical and biological influences to modify the behaviour of the cell phase and so forth.

In this context, the polymeric structure can further comprise one or more active agents. As used herein, an “active agent” is any agent capable of altering, modifying or otherwise interacting with the surrounding environment once brought into direct or indirect contact with it. An active agent can be any agent having the ability to bring about chemical reactions or physical state changes. Suitable agents to be used in the frame of the present invention are for instance, and without limitation, imaging or contrast agents, markers or dyes such as fluorescent dyes, bioactive agents, magnetically or optically active substances, organic compounds, inorganic compounds and/or elements (such as e.g., gold particles), coating substances and/or precursors thereof, viruses, cells including fungi, bacteria or spores thereof, or food substances including seeds or probiotics.

In the frame of the present disclosure, the expression “bioactive molecule”, as well as “bioactive compound”, “active agent”, “bioactive agent” or “therapeutic agent”, refers to any agent that is biologically active, i.e. having an effect upon a living organism, tissue, or cell. The expression is used herein to refer to a compound or entity that alters, inhibits, activates, or otherwise affects biological or chemical events. Bioactive compounds according to the present disclosure can be small molecules or preferably macromolecules, including recombinant ones.

Exemplary therapeutic agents include, but are not limited to, a growth factor, a protein, a peptide, an enzyme, an antibody or any derivative thereof (such as e.g. multivalent antibodies, multispecific antibodies, scFvs, bivalent or trivalent scFvs, triabodies, minibodies, nanobodies, diabodies etc.), an antigen, a nucleic acid sequence (e.g., DNA or RNA), a hormone, an anti-inflammatory agent, an anti-viral agent, an anti-bacterial agent, a cytokine, an oncogene, a tumor suppressor, a transmembrane receptor, a protein receptor, a serum protein, an adhesion molecule, a lypidic molecule, a neurotransmitter, a morphogenetic protein, a differentiation factor, an analgesic, organic molecules, metal particles, radioactive agents, polysaccharides, a matrix protein, a cell, and any functional fragment or derivative of the above, as well as any combinations thereof. For “functional fragment” is herein meant any portion of an active agent able to exert its physiological/pharmacological activity. For example, a functional fragment of an antibody could be an Fc region, an Fv region, a Fab/F(ab')/F(ab')2 region and so forth.

In a particular embodiment, at least part of the plurality of polymeric cells comprised within the polymeric structure of the invention are labelled with a marker or a dye, such as a fluorescent dye.

The invention also provides pharmaceutical compositions comprising the polymeric structure of the invention. These compositions may, optionally and additionally, comprise a pharmaceutically acceptable carrier, excipient and/or diluent. As used herein, “pharmaceutically acceptable carrier” includes any and all solvents, dispersion media, coatings, antibacterial and antifungal agents, isotonic and absorption delaying agents and the like, that are physiologically compatible. Examples of suitable pharmaceutical carriers are well known in the art and include sodium chloride solutions, phosphate buffered sodium chloride solutions, water, emulsions, such as oil/water emulsions, various types of wetting agents, sterile solutions, organic solvents and so forth. The pharmaceutically acceptable carrier suitably contains minor amounts of additives such as substances that enhance isotonicity and chemical stability. Such materials are non-toxic to recipients at the dosages and concentrations employed, and include buffers such as e.g. phosphate, citrate, succinate, acetic acid, hyaluronic acid and other organic acids or their salts; antioxidants such as ascorbic acid; low molecular weight (less than about ten residues) (poly)peptides, e.g., polyarginine or tripeptides; proteins such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

Besides, other potential applications can be envisaged for the polymeric structures herein developed. For instance, structured hydrogels can potentially be used as high throughput screening platform. Different polymeric cells can be loaded with different reagents and labels such that they can be differentiated. This would enable studies of influence of these reagents for example on cell behaviour or on the binding of proteins on very small scales. Optionally these hydrogels can be made from polymeric cells loaded with different reagents that are either released over time (through diffusion) or upon exposure to an external stimulus such as mechanical compression.

Structured hydrogels can also be made from monodisperse polymeric cells whose mechanical properties can be tuned. These structures have the potential to absorb a high energy density if they are made from flexible capsules because they can be reversibly compressed, thus working as damping systems.

Another aspect of the present invention relates to the use of the polymeric structure of the invention as identifying tags in the form of non-printed, physical data matrix tags (e.g., QR codes). Two-dimensional codes can be found everywhere in everyday life.

This technology conveniently and quickly allows to recognise products or services information. The diffusion of devices such as smart phones permits to easily access and read relevant information about goods, so that consumers no longer need specialized equipment and mass diffusion can be achieved.

A (visual) data matrix code is a two-dimensional barcode consisting of black and white “cells” or modules arranged in either a square or rectangular pattern, also known as a matrix, encoding information. The information to be encoded can be text or numeric data, and the length of the encoded data depends on the number of cells in the matrix. They are typically implemented as grids designed to be scanned by an optical scanner and subsequently decoded to determine the information contained in it. Quick Response codes (QR codes) are a particular class of data matrix codes, that shows an advantage in terms of e.g. high-speed and all-direction (360 degrees) accessibility, and capability of representation of Chinese characters, rendering QR code wide applicability in various fields. It comprises a square array of a series of small square message blocks, in which “0” or “1” are represented through alternation of bright and dark blocks.

Data matrices can contain varying amounts of information, for example, from a few characters to thousands of characters, may contain a web address, and can be placed directly on an item. For example, a data matrix can be silkscreened directly onto a product. It can also be located directly on a product by moulding the matrix onto the product or etching it onto a product surface.

Data matrices can also contain identification information. For instance, it can contain information identifying a particular item or product associated with the data matrix. A particular brand and model of an item can be identified by information contained in a related matrix that may be located on the product or product packaging. A data matrix can also identify a particular version of a product or further information such as date of manufacture, version number, expiration date, etc., or any other variant of a product.

Based on the above considerations, it is proposed a physical micro-tag article in which no design step for the tags is involved. All known approaches for creating data matrices are deterministic (that is, the produced pattern is set before its implementation), and thus the resulting matrix patterns can be easily reproduced. On the contrary, the present invention proposes a new set-up based on a stochastic process, wherein the production of the data matrix derives from a well-established procedure giving unpredictable results, and thus random and unique patterns analysable only a posteriori.

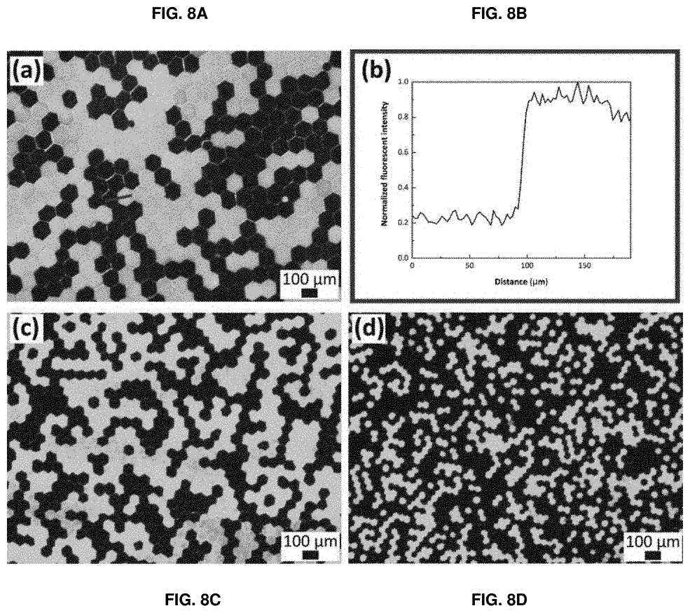

The polymeric structure of the invention can be made from particles with different properties (colour, stiffness, surface roughness, sizes) that are randomly assembled (see, for example FIGS. 8A-8D). As a result, chances to generate two times exactly the same patterned hydrogels are very low. This opens up possibilities to use these polymeric materials such as hydrogels as uniquely identifying tags that must first be associated to the product they are labelling but can thereafter be used as an identifying tag. Moreover, hydrogels have the potential to be made food-grade and most likely also transparent, such that they could also be used as labels of food.

Accordingly, a further aspect of the present invention relates to a physical, non-printed polymeric data matrix tag, characterized in that it comprises a polymeric structure of the invention shaped as a flat sheet comprising a non-flat surface, and/or in which part of the plurality of the polymeric cells are labelled with a marker or a dye, such as a fluorescent dye.

The so-obtained data matrix tags can be machine-readable using low cost instruments, and can be batch produced in very large quantities at a contained cost. The further possibility of miniaturizing the tags makes them easy to be integrated in a larger identification system with multiple security layers, being further easily hidden if needed such that it could result unnoticeable or at least barely noticeable.

As outlined above, the surface roughness of the polymeric structure can be modified. In an embodiment, the polymeric cells may comprise a semi-spherical surface. The semi-spherical surface may be a dome. The size of the dome may be tuned depending on the desired degree of surface modification.

The polymeric structures according to the invention have been developed by exploiting a new manufacturing method taking advantage from a tailored and elegant mix of materials' choice, polymerization kinetics and, in certain preferred embodiments, the microfluidic technology.

Accordingly, an aspect of the invention relates to a method for producing a polymeric structure comprising a plurality of spherical or polyhedral polymeric cells bound through their sidewalls via a connection element, wherein the polymeric cells and the connection element have a different Young's modulus, said method comprising the steps of:
a) providing a plurality of spherical or polyhedral polymeric cells disposed on a support substrate;
b) providing a connection element between the sidewalls of the polymeric cells; and
c) allowing a crosslinking reaction between the polymeric cells and the connection element by providing a crosslinking trigger.

In one embodiment, the step of providing a plurality of spherical or polyhedral polymeric cells disposed on support substrate in a packed arrangement comprises:
a) providing a plurality of drops comprising chemical precursors of a polymeric matrix composing the polymeric cells, said drops being arranged into a non-chemically bound packed arrangement on said support substrate; and
b) starting a polymerization reaction of the polymeric matrix composing the polymeric cells.

Said polymerization reaction can be brought about by providing a crosslinking trigger, as will be detailed later on.

In these embodiments, a plurality of polymeric cells in a polymerized form can be arranged in a substrate comprising for instance a preformed pattern such as a grid to dispose and assemble the cells or precursors thereof in a packed, ordered fashion along or within a substrate support, so to facilitate the final aspect of the polymeric structure. Said grid can work as a connection element by itself, or can lately be removed at any point of the manufacturing process.

In one embodiment, the step of providing a connection element between the sidewalls of the polymeric cells comprises:
a) disposing a liquid or semi-solid connection element; and
b) optionally allowing solidification or polymerization of said liquid or semi-solid connection element.

Said solidification or polymerization reaction can be brought about by providing a crosslinking trigger, as will be detailed later on. This embodiment can be carried out in circumstances when the connection element is not already in a solidified/polymerized form. The liquid or semi-solid connection element is disposed between the polymeric cells, and possibly below and/or above these latter.

In one particular embodiment, the method comprises the steps of:
a) providing an emulsion comprising:
   a first phase comprising a plurality of drops of chemical precursors of a polymeric matrix dispersed into an immiscible second phase; and
   a crosslinker;
b) disposing said emulsion into a support so that the drops assemble into a non-chemically bound packed arrangement;
c) starting a polymerization reaction of the polymeric matrix by providing a crosslinking trigger; and
d) during the polymerization reaction, and before the polymerization reaction is complete, allowing the removal of the second phase.

Whenever needed, the above method further comprises a step of continuing the polymerization reaction up to the formation of the polymeric connection element.

The starting emulsion comprises a first phase and a second phase; as per the emulsion definition, the two phases are not or minimally miscible between them. In an emulsion, one liquid (the dispersed phase) is dispersed in the other (the continuous phase). Although the terms colloid and emulsion are sometimes used interchangeably, emulsion should be used when both phases, dispersed and continuous, are liquids.

However, in the frame of the present disclosure, the terms “colloid” or “colloidal solution” could be used to indicate an emulsion, and can even be used in its proper sense of a mixture in which one substance of microscopically dispersed insoluble particles (the dispersed phase or first phase) is suspended throughout another substance (the continuous phase or second phase).

As said, the two phases are not or minimally miscible. In this context, the first phase can be an aqueous phase or aqueous solution, and the second phase an organic or non-polar solution, or vice-versa. An “aqueous solution” is a solution in which the solvent is substantially made of water. In the frame of the present disclosure, the term “aqueous” means pertaining to, related to, similar to, or dissolved in water. The expression aqueous solution in the frame of the present disclosure also includes highly concentrated and/or viscous solutions such as for instance syrups (i.e., saturated water/sugars solutions) and the like, in which the water content is e.g. less than 5% weight of the total solution weight. A “non-polar solution” is a solution in which the solvent is a non-polar compound. Non-polar solvents are intended to be compounds having low dielectric constants and that are not miscible with water. A non-exhaustive list of non-polar solutions can comprise for example solutions comprising oils, benzene, carbon tetrachloride, chloroform, diethyl ether, xylene, toluene, ethanol, hexanol, heptanol, decanol, dodecanol, hydrocarbon-based solutions (e.g. hexane, cyclohexane, n-octane, isooctane, decane, hexadecane and the like), fluorophilic solvents, ethyl acetate, silicon oils, mineral oils, oils used for food and so forth. An “oil” is any non-polar chemical substance that is a liquid at ambient temperatures and is both hydrophobic and lipophilic. A fluid material is also intended to comprise any fluid material comprising a gas dispersed within, such as e.g. liquid-gas solutions.

Two liquids can form different types of emulsions. As an example, oil and water can form, first, an oil-in-water emulsion, wherein the oil is the dispersed phase, and water is the dispersion medium. Second, they can form a water-in-oil emulsion, wherein water is the dispersed phase and oil is the external phase. Multiple emulsions are also possible, including a “water-in-oil-in-water” emulsion and an “oil-in-water-in-oil” emulsion.

As used herein, the term “drops” may be particles of between 10 nm and 10 mm in size. In the frame of the present disclosure, and for the sake of clarity and conciseness, the term is used indifferently to intend several kinds of particles such as microparticles, (micro)capsules, beads, vesicles, grains and the like. A “microcapsule”, also referred to herein as “core-shell capsule” is a micrometer-scale particle such as for instance gas bubbles or liquid drops surrounded by a solid, liquid, or otherwise fluid shell. Drops according to the invention can have a diameter typically in the range of 1 to 1000 μm, such as for instance 50, 100, 200 or 500 μm. Particularly preferred is between 1 μm and 200 μm.

A core-shell capsule is a substantially spherical micro- or nanocapsule characterized in that it is hollow in its inner core, and comprises in said core a fluid material which is encapsulated by the outer shell. It is a particle composed of a drop (the core) contained in a second, larger drop (the shell membrane) made of an immiscible, or partially miscible fluid, or even a solid or semi-solid material such as a gel. The second, larger drop typically contains the chemicals that are used to optionally solidify the shell. However, certain chemicals can also be contained in the smaller drop, which forms the core of the double emulsion, or in the outermost phase, where the drops are dispersed in. The core of the capsule or double emulsion is substantially made of an aqueous solution. These capsules or double emulsions are dispersed in a continuous aqueous phase (the water-in-oil-in-water type, or W-o-W). Alternatively, the core of the capsule or double emulsion is composed of an oil, the larger drop is made of an aqueous phase and the double emulsion drops are dispersed within a continuous oil (the oil-in-water-in-oil type, or O-w-O). In still alternative embodiments, the double emulsion can be an oil-oil-oil emulsion (e.g. hydrocarbon-fluorinated-hydrocarbon oil) or water-water-oil emulsion where two aqueous phases are made immiscible through the addition of high concentrations of immiscible water-soluble polymers. They are also referred to herein as “double emulsion capsule”.

Emulsion drops or core-shell capsules according to the invention offer the possibility of including within the inner core one or more active agents. This is of particular interest for what concerns their application, such as e.g. biomedical, cosmetic, agriculture, coating or food ones. In some embodiments of the invention, drops comprise active agents such as bioactive agents as previously described. In additional or alternative embodiments of the invention, drops comprise a marker or a dye to label the polyhedral polymeric cells, such as a fluorescent marker or dye, for example fluorescein or derivatives thereof as fluorescein isothiocyanate (FITC).

Drops according to the invention comprises or substantially consist of chemical precursor(s) or the polymeric matrix composing the polymeric structure of the invention. Said precursor(s) can be monomers, oligomers or even polymers that are subsequently polymerized during the production process. Chemical precursors of the polymeric matrix according to the invention may comprise one or more compounds or monomers thereof or oligomers thereof selected from a non-exhaustive list comprising natural polymeric materials (i.e., non-synthetic polymers, polymers that can be found in nature) and/or polymers derived from Extra Cellular Matrix (ECM) as gelatin, elastin, collagen, agar/agarose, chitosan, fibrin, proteoglycans, a polyamino-acid or its derivatives, preferably polylysin or gelatin methyl cellulose, carbomethyl cellulose, polysaccharides and their derivatives, preferably glycosaminoglycanes such as hyaluronic acid, chondroitinsulfate, dermatansulfate, heparansulfate, heparine, keratansulfate or alginate, as well as any derivative thereof, fragment thereof and any combination thereof;

thermoset materials such as alkyds, epoxies, phenolics (e.g., Bakelite), polyimides, formaldehyde resins (e.g., urea formaldehyde or melamine formaldehyde), polyester thermosets, unsaturated polyesters, polyurethane, bis-maleimides (BMI), silicone materials such as polydimethylsiloxane (PDMS) and any combination thereof;

and other materials such as poly(lactic-co-glycolic acid), lactide and glycolide polymers, caprolactone polymers, hydroxybutyric acid, polyanhydrides, polyesters, polyphosphazenes, polyphosphoesters and poly(glycerol sebacate acrylate), polypropylene, polypropylenoxide or their derivatives, polymethylenoxide or its derivatives, polyethylene or its derivatives such as polyethylene glycole (PEG), polyethylenoxide or their derivatives, polyacrylate or its derivatives, poly(vinyl alcohol) (PVA) and copolymers, poly(vinylpyrrolidone) (PVP) and combinations thereof; as well as any combination of the foregoing.

The emulsion, as well as any precursor of the polymeric cells and/or the connection element, further comprises a crosslinking agent, also referred herein as “curing agent” or “crosslinker”. It is appreciated that the crosslinking agent may be employed to chemically cross link the cross-linkable monomers, oligomers and/or polymers of the chemical precursor(s) of the polymeric matrix. Suitable crosslinking agents can comprise for instance 1,4-Cyclohexanedimethanol divinyl ether, di(ethylene glycol) diacrylate, di(ethylene glycol) dimethacrylate, N,N′-(1,2-Dihydroxyethylene)bisacrylamide, divinylbenzene, p-Divinylbenzene, ethylene glycol diacrylate, ethylene glycol dimethacrylat, 1,6-Hexanediol diacrylate, 4,4′-Methylenebis(cyclohexyl isocyanate), 1,4-Phenylenediacryloyl chloride, Trimethylolpropane ethoxylate triacrylatepoly(ethylene glycol) diacrylate, poly(ethylene glycol) dimethacrylate, tetra(ethylene glycol) diacrylate or tetraethylene glycol dimethyl ether. However, in certain embodiments, the crosslinking agent can be nanoparticles, functionalized surfactants or even a portion or a chemical group present in the monomers, oligomers and/or polymers of the chemical precursor(s) of the polymeric matrix. As a way of example, acrylate groups can be the reactive crosslinking groups present on the chemical precursor(s) of the polymeric matrix. Yet further crosslinking agents include ions. Examples of suitable ions include but are not limited to Fe²⁺, Ca²⁺ or Ni²⁺.

In some embodiments, the emulsion of the invention, as well as any precursor of the polymeric cells and/or the connection element, can further comprise a photoinitiator. A “photoinitiator” is a molecule that creates reactive species (free radicals, cations or anions) when exposed to an electromagnetic radiation such as UV or visible light. Example of suitable visible or ultraviolet light-activated photoinitiator includes ITX 4-Isopropyl-9-thioxanthenone, Lucirin TPO 2,4,6-Trimethylbenzoyl-diphenyl-phosphineoxide, Irgacure 184 1-Hydroxy-cyclohexyl-phenyl-ketone, Irgacure 2959 1-[4-(2-Hydroxyethoxy)-phenyl]-2-hydroxy-2-methyl-1-propane-1-one, Irgacure 819 Phosphine oxide, phenyl bis (2,4,6-trimethyl benzoyl), LAP lithium phenyl-2,4,6-trimethylbenzoylphosphinate, Riboflavin 7,8-dimethyl-10-((2R,3R,4S)-2,3,4,5-tetrahydroxypentyl) benzo [g] pteridine- 2,4 (3H,10H)- dione, Rose Bengal 4,5,6,7-tetrachloro-2′,4′,5′,7′-tetraiodofluorescein, PL-BDK Benzil dimethyl ketal, PL-CPK 1-hydroxy-cyclohexylphenyl-ketone, PL-HMPP 2-hydroxy-2-methyl-1-phenyl-1-propanone, Camphorquinone, 3-(4-Quantucure BPQ benzoylphenoxy)-2-hydroxy-N,N,N-trimethyl-1-propanaminium-chloride, APi-180 hydroxyalkylpropanone, bisacylphosphineoxide- or monoacylphosphineoxide-based initiators.

Once the starting emulsion is provided, the method according to the invention foresees a second step of disposing said emulsion into a support so that the drops assemble into a non-chemically bound packed arrangement, said support being possibly but not exclusively non wettable by the first phase. As will be detailed later on in the Examples, the use of a support substrate non wettable by the first phase of the emulsion facilitates the polymerization of the polymeric connection element so that the adjacent polymeric cells are connected between them through their sidewalls. In particular, for “support non wettable by the first phase” is herein meant that the physico-chemical properties of the support substrate on/in which the emulsion is disposed are such to impede adhesion of the first phase on it. For instance, in cases where the first phase is an aqueous, polar phase, the support will substantially consist of a hydrophobic material such as PDMS, whereas in cases where the first phase is an organic phase, the support will substantially consist of a hydrophilic material such as glass.

In a third step, the method foresees starting a polymerization reaction of the polymeric matrix by providing a crosslinking trigger. Said trigger can be for example a thermal and/or light trigger, such as heat, infrared radiation or, UV light. The choice of the crosslinking trigger depends on several factors such as the polymeric matrix, the elected crosslinker or the intended kinetics of the polymerization reaction, just to cite a few. A person skilled in the art would easily derive the crosslinking trigger to be used on a case-by-case basis, depending on the needs and/or circumstances. In embodiments where UV light is used, the irradiation time can span from few seconds, such as between 3 and 15, up to one minute, depending on the light intensity of the UV light source and the irradiance. Accordingly, the distance of irradiation can vary from e.g. 0.5 to 10 cm, such as between 1 and 3 cm. The crosslinking triggers used in this embodiment of the invention are however the same used for all other embodiments, and will not be re-listed elsewhere for the sake of conciseness.

During a fourth step of the method, the removal of the second phase is allowed and possibly facilitated. This step is required to bring the drops into close contact, and is performed during the polymerization reaction, and before the polymerization reaction is complete. In certain circumstances, a slow and continuous removal of the second phase from the emulsion brings to a close packing of drops in the first phase up to the rupture of the drops. In these embodiments, the rearrangement of the polymeric matrix is favoured towards a crystalline lattice such as a hexagonal, honeycomb-like lattice. In a last optional step, the polymerization reaction is allowed to continue up to the formation of the polymeric connection element whenever needed.

Additionally, the method can further comprise a step of labelling at least part of the plurality of drops with a marker or a dye, such as a fluorescent dye as fluorescein or derivatives thereof (e.g. fluorescein isothiocyanate). This optional step is preferably performed before the polymerization process starts, and even more preferably a marker or a dye is included as of the beginning into the first phase and within the drops, in embodiments where those drops are present. Metallic, ferromagnetic or superparamagnetic micro/nanoparticles could also be envisaged as a marker, particularly for separation and/or purification purposes, and/or for arranging the polymeric cells within the material by exploiting for instance a magnetic field. Further possible uses include sensing or actuation purposes. An example micro/nanoparticle is gold. Another example is the iron oxide nanoparticle.

In a particular embodiment of the invention, the step of providing an emulsion is performed through a microfluidic device. A “microfluidic device”, “microfluidic chip” or “microfluidic platform” is generally speaking any apparatus which is conceived to work with fluids at a micro/nanometer scale. Microfluidics is generally the science that deals with the flow of liquids inside channels of micrometer size. At least one dimension of the channel is of the order of a micrometer or tens of micrometers in order to consider it microfluidics. Microfluidics can be considered both as a science (study of the behaviour of fluids in micro-channels) and a technology (manufacturing of microfluidics devices for applications such as lab-on-a-chip). These technologies are based on the manipulation of liquid flow through microfabricated channels. Actuation of liquid flow is implemented either by external pressure sources, external mechanical pumps, integrated mechanical micropumps, hydrostatic pressures or by combinations of capillary forces and electrokinetic mechanisms.

The microfluidic technology has found many applications such as in medicine with the laboratories on a chip because they allow the integration of many medical tests on a single chip, in cell biology research because the micro-channels have the same characteristic size as the cells and allow such manipulation of single cells and rapid change of drugs, in protein crystallization because microfluidic devices allow the generation on a single chip of a large number of crystallization conditions (temperature, pH, humidity . . . ) and also many other areas such as drug screening, sugar testers, chemical microreactor or micro fuel cells.

In the frame of the present invention, a microfluidic device can be easily adapted to work with fluid volumes spanning from millilitres down to femtoliters, and the dimensions can be adapted accordingly to have channels within the millimetre scale, without substantially departing from the teaching of the invention.

Generally speaking, a microfluidic device or system is intended for production of particles or droplets comprising or substantially composed of a fluid material or combinations of more fluid materials. In a typical scenario, a microfluidic device comprises one or more reservoirs, or is fluidically connected to one or more reservoirs, containing fluid material(s) composing the first phase (also called “dispersed phase”), and one or more reservoirs containing a substantially immiscible second phase, also called “continuous phase”. In this context, “substantially immiscible” means that vast majority of the first phase fluid, i.e. at least 90% thereof, is not solubilized by the continuous phase fluid. The wording “at least partially miscible” can be used interchangeably. This is basically linked to the method of production of the droplets, exploiting the effect of the continuous phase fluid on the dispersed phase.

The four most common strategies for obtaining droplets in a microfluidics setting are the use of step-junction, T-junction, Y-junction or flow focusing geometries. The step-emulsification exploit the transition from confined to unconfined flow for micro-droplet generation. A narrow rectangular inlet channel leads to a wide and deep reservoir. The dispersed phase (non-wetting the channel walls) expands to form a tongue which grows until it reaches the step-like formation at the entrance to the reservoir. At the step the tongue expands into unconfined spherical droplet that pinches-off from the tongue.

In a typical T-junction configuration, the two immiscible phases meet face to face and then flow through orthogonal channels, forming droplets by squeeze where they meet depending on the volumetric rates of flow of the two immiscible fluids. A Y-junction configuration is a modification of the T-junction setting wherein the two feeding microchannels (one for the continue phase and one for the dispersed phase) meet with a relative inclination angle different from 0°.

In the flow focusing technique, the continuous phase fluid flanks or surrounds the dispersed phase, exerting pressure and tangential viscous stress over this latter so as to give rise to droplet or bubble break-off through capillary instability in the vicinity of an orifice through which both fluids are extruded. As it will be evident, the principle may be extended to two or more coaxial fluids, and gases and liquids may be combined, depending on the needs. All the above described microfluidic chip configurations for obtaining micro/nanodroplets are well known techniques readily available to a skilled person, and a complete review thereof can be found in Gu et al. (Int. J. Mol. Sci. 2011, 12, 2572-2597).

The invention may now be described by reference to the following non-limiting examples.

### EXAMPLES

Microfluidic Device Fabrication

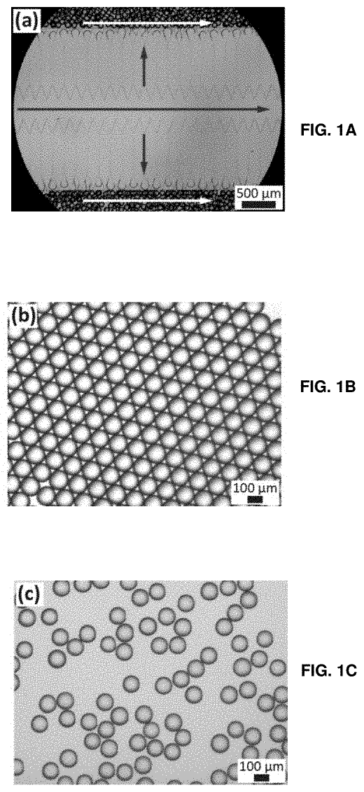

Drops are produced in microfluidic millipede devices made of poly(dimethyl siloxane) (PDMS, Sylgard 184, Dow Corning) using soft lithography. These devices consist of a central channel for the inner phase and on each of the two long sides of the central channel there is a channel for the oil phase. The central channel is connected to the two outer channels through 300 individual drop makers. The nozzle of the drop makers opens in a triangular way, as shown on the optical microscopy image in FIG. 1A. For a proper functioning of the device, the channel walls must be non-wetting for the inner phases. Therefore, the channels have been treated with an HFE 7500-based solution containing 1% (v/v) trichloro(1H,1H,2H,2H-perfluorooctyl)silane (Sigma-Aldrich).

Drops Production

An aqueous solution containing 50% (w/w) poly(ethylene glycol) diacrylate (PEG700-DA, Mw˜700 Da, Sigma-Aldrich), a monomer, and 2% (w/w) 2-hydroxy-2-methylpropiophenone (97%, Sigma-Aldrich), a photoinitiator, is used as the first, inner phase. To produce fluorescently labelled hydrogels, 0.1 mg/mL fluorescein isothiocyanate-dextran (FITC-Dextran, Mw˜150,000 Da, Sigma-Aldrich) was added to the aqueous first phase containing the monomers and photoinitiator. A perfluorinated oil (Novec HFE 7500, 3M) with 1% (w/w) fluorinated surfactant is used as an outer, continuous phase. The two phases are injected into the millipede devices using volume controlled syringe pumps (Cronus Sigma 1000, Labhut, UK). Drops are collected in a glass vial that is wrapped in aluminium foil to protect collected drops from light exposure.

Assembly of Structured Hydrogel Sheets from Drops

The density of water is 1.6 times lower than that of the oil such that drops cream inside the collection vial. Part of the oil was removed to increase the drop concentration before this emulsion is deposited onto a clean glass slide or into a 25 mm long, 4 mm wide and 120 μm deep PDMS trough. After the drops self-assembled into a monolayer, excessive drops are removed using a micropipette. While the oil evaporates, drops attain a hexagonal prismatic shape. Subsequently, they were illuminated with UV light whose wavelength ranges from 320 nm to 500 nm (Ominicure S 1000, Lumen Dynamics, Canada) for 15 s, keeping the distance between the sample and the UV light source constant at 3 cm.

Assembly of Structured Hydrogels from Hydrogel Microparticles

To convert drops into hydrogel microparticles, they are illuminated with UV light. The polymerized hydrogel microparticles are washed three times with 2,2,3,3,4,4,4-heptafluoro-1 butanol (TCI) to remove the surfactant and subsequently dispersed into an aqueous solution. 20 vol % ethanol (Sigma-Aldrich) was then added to the aqueous dispersion containing the hydrogel microparticles to lower the surface tension, thereby facilitating the spreading of the emulsion on the PDMS substrate; this is required to produce monolayers of microparticles. A few drops of this dispersion were deposited onto a PDMS substrate and let the particles sediment. The sample was subsequently vortexed to agitate the particles, thereby facilitating their arrangement into a hexagonal close packed structure.

Characterization

Interfacial tension: The contact angle between the aqueous solution containing PEG-DA and the substrate as well as the interfacial tension between the PEG-DA solution and the oil is measured using the drop shape analyzer (DSA25, Kruss, Germany).

Microscopy: Samples are visualized with the optical microscope (Eclipse TS 100 and Ti-E, Nikon, Japan). Additionally, some samples are characterized using a scanning electron microscope (SEM, XLF30-FEG, FEI, USA). SEM images are acquired at an acceleration voltage of 3 kV using a secondary electron detector. To make the samples electrically conductive, they are coated with a 30 nm thick gold film.

Mechanical properties: The Young's modulus of the hydrogel sheets is measured using tensile tests (MiniMat 2000, Rheometric Systems). Samples are dried in air at room temperature for at least 2 days before the measurements are conducted. Sample sheets are immobilized with two clamps that are coated with a 1 mm thick PDMS layer to avoid sample damage. The applied force was measured as a function of the strain and the stress calculated by dividing the force by the cross section of the sample, which was quantified by measuring its width using optical microscopy and its thickness using a digital micrometer.

To produce aqueous drops that display a narrow size distribution, a microfluidic millipede device that contains 550 parallelized nozzles was used, which have triangular shapes, as shown in FIG. 1A. The employed device had 40 μm tall nozzles to produce 119 μm diameter aqueous drops. Their coefficient of variation, defined as the standard deviation of their size distribution divided by the mean diameter, is as low as 2.5%, as shown in the optical micrograph in FIG. 1 B.

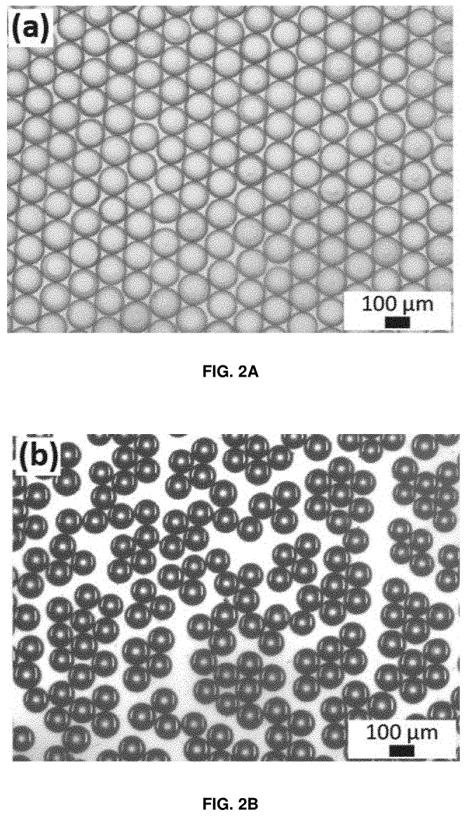

Two dimensional colloidal crystals composed of hard spheres are usually made by spreading a dispersion containing monodisperse colloids onto a solid substrate and letting the particles arrange into the energetically most favourable crystal structure. To mimic this process, PEG-DA-containing drops were converted into hydrogel microparticles by exposing them to UV light to initiate the polymerization reaction. The resulting microparticles display a narrow size distribution, as shown in FIG. 1C. The dispersion was then deposited onto a PDMS substrate to let the hydrogel particles sediment and facilitate their re-arrangement by agitating the substrate using a vortex mixer. If sufficiently agitated, hydrogel microparticles assemble into the thermodynamically most favorable hexagonally close packed structure, as shown FIG. 2A. However, the crystal structure becomes defective upon evaporation of the water because the particles shrink without being connected to each other, as shown in the optical micrograph in FIG. 2B.

To build macroscopic materials from individual microparticles, adjacent particles must be connected to each other. This can be achieved by back-filling the structured hydrogels with a second matrix, using strategies employed for the production of inverse opals and photonic crystal biosensor. However, these hydrogel microparticles can be infiltrated with the matrix used to back-fill the substrate, which could result in a change in the structure and properties of the particles. To overcome this limitation, adjacent particles were covalently connected using reactive groups that are present at their surfaces. Monomer-containing drops were deposited on a substrate where they spontaneously assemble into a hexagonally close packed 2D structure.

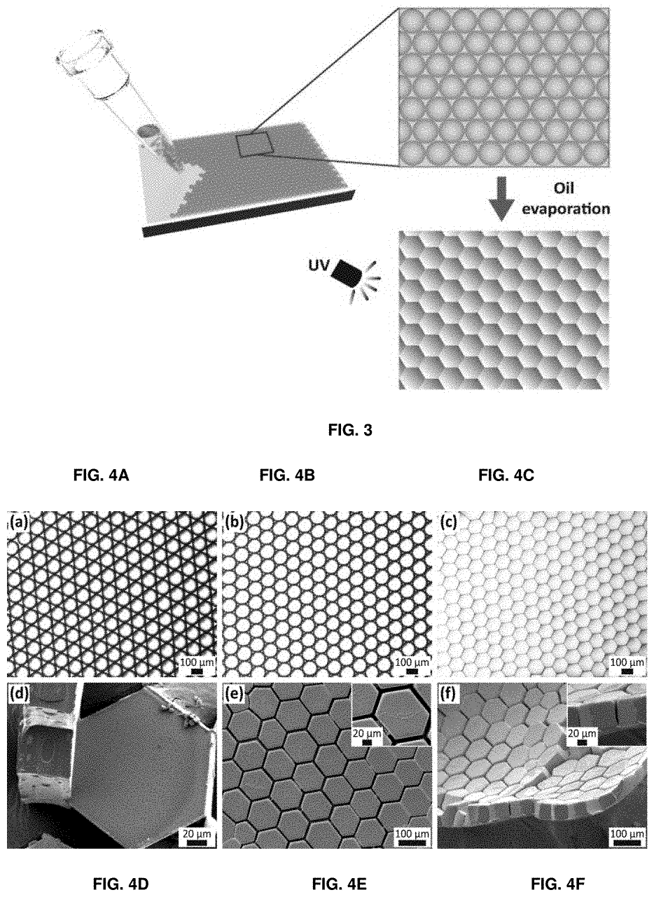

They were subsequently illuminated with UV light to initiate the polymerization reaction of PEG-DA, as shown schematically in FIG. 3. During the assembly of drops and the polymerization of PEG-DA, the oil evaporates and when the volume of oil that remains on the substrate is too small, drops start to rupture. If drops rupture before all the monomers are consumed, it is expected some of the unreacted monomers to form covalent crosslinks between adjacent particles if they are located at the particle surface. Unreacted monomers are preferentially located at the drop liquid-liquid interface and hence end up at the particle surface if the interfacial tension between the monomers and the oil is below that of the hydrogel particles and the oil. In the implemented embodiment, the interfacial tension between the PEG-DA monomers and HFE 7500 is 15.7±0.1 mN/m; it is much lower than that between hydrogels and HFE 7500, which we approximate to be that of water/HFE 7500 interfaces, 41.6±0.6 mN/m. Hence, the hydrogel microparticles are expected to be surrounded by monomers that can covalently link adjacent microparticles.

To covalently link adjacent particles, in the exemplary embodiment herein reported drops must rupture before all the monomers are polymerized. The rate at which drops rupture depends on the oil evaporation rate, and thus is in a first approximation independent of the UV light intensity, if the heating of the UV source is neglected. By contrast, the rate at which monomers are crosslinked depends on the UV light intensity. Hence, if the UV light intensity is reduced to sufficiently low values, thereby slowing down the initiation of the polymerization reaction, it is expected that drops rupture before all the monomers are consumed. To test this expectation, the inventors deposited drops on a glass slide and let the oil evaporate. Once the majority of the oil is evaporated, drops deform to attain a hexagonal prismatic shape, as shown in the time-lapse optical micrographs in FIGS. 4A-4C. If they are subsequently illuminated for 15 s with UV light that is 1 cm apart from the sample, monomers polymerize before drops rupture such that individual hexagonal prismatic microparticles result, as shown in the SEM micrograph in FIG. 4D. By contrast, if one increases the distance between the UV source and the sample to 3 cm, we can easily detach a connected film composed of the hexagonal prismatic microparticles from the glass slide, as shown in FIG. 4E. However, the microparticles are not connected through any of their sidewalls. Instead, they are connected through a thin film at the bottom of this structure, as shown in the SEM micrograph in FIG. 4F. It was assumed that the film formation was due to the contact angle between the monomer solution and the glass slide, which is as low as 14°±2°. Hence, the monomer containing aqueous solution wets the glass slide as soon as drops rupture. Upon exposure to UV light, the monomers contained in these thin films polymerize, thereby forming a connecting hydrogel film at the bottom of the particle array. While these structures are mechanically sufficiently stable to be detached from the glass slide, the sheet tends to roll because the stress in the connecting bottom layer is much higher than that at the top of the sheet. This inhomogeneous stress distribution makes the handling of these films cumbersome.

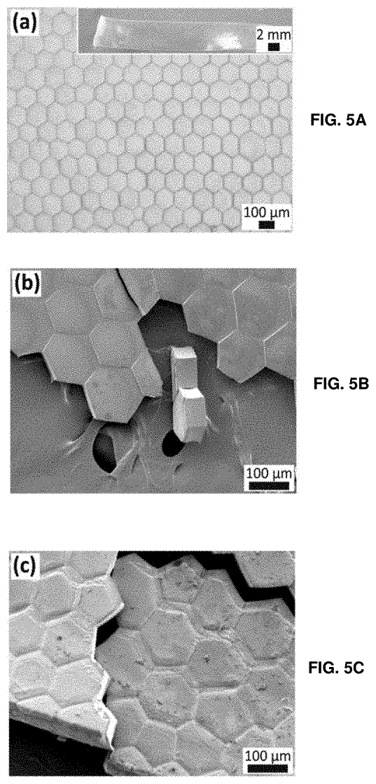

Monomers form thin films at the bottom of the particle arrays, if they wet the substrate surface. To build sheets that do not roll when detached from the substrate, this film formation must be prevented. Therefore, drops were deposited onto a hydrophobic substrate made of poly(dimethyl siloxane) (PDMS); the contact angle between the monomer solution and PDMS is 87°±4°. After drops attain a hexagonal prismatic shape, they were illuminated with UV light for 15 s. The resulting macroscopic sheets are again mechanically sufficiently stable to be detached from the substrate, as shown in FIG. 5A. Sheets formed on PDMS substrates remain flat even after they have been detached from the substrate, indicating that the stress distribution along the height of these sheets is more homogeneous. Indeed, there is no connecting film at the bottom of the particle assembly, as shown in the SEM images in FIGS. 5B-5C, indicating that adjacent particles are connected through their sidewalls.

The type and density of the connections between adjacent particles strongly influence the mechanical properties of the resulting structured hydrogel sheets. To compare the mechanical properties of the structured sheets to those of bulk hydrogels, tensile tests on dried samples were performed. The Young's modulus of unstructured sheets is 7.7±0.7 MPa, as detailed in Table 1, well in agreement with that in bulk hydrogels made of PEG-DA of similar molecular weights.

TABLE 1
Young's modulus of hydrogel sheets as a function
of the concentration of monomers contained in drops,
their composition, and the hydrogel sheet structure

| | M_{w} | | E |
|---|---|---|---|
| concentration | of PEG | structure | (MPa) |
| 50% | 700 | unstructured | 7.7 ± 0.7 |
| | | structured | 12.0 ± 1.0 |
| | 575 | unstructured | 9.6 ± 1.0 |
| | | structured | 12.0 ± 1.5 |
| 40% | 700 | unstructured | 6.3 ± 0.5 |
| | | structured | 9.8 ± 0.3 |
| | 575 | unstructured | 6.8 ± 0.8 |
| | | structured | 9.6 ± 1.2 |

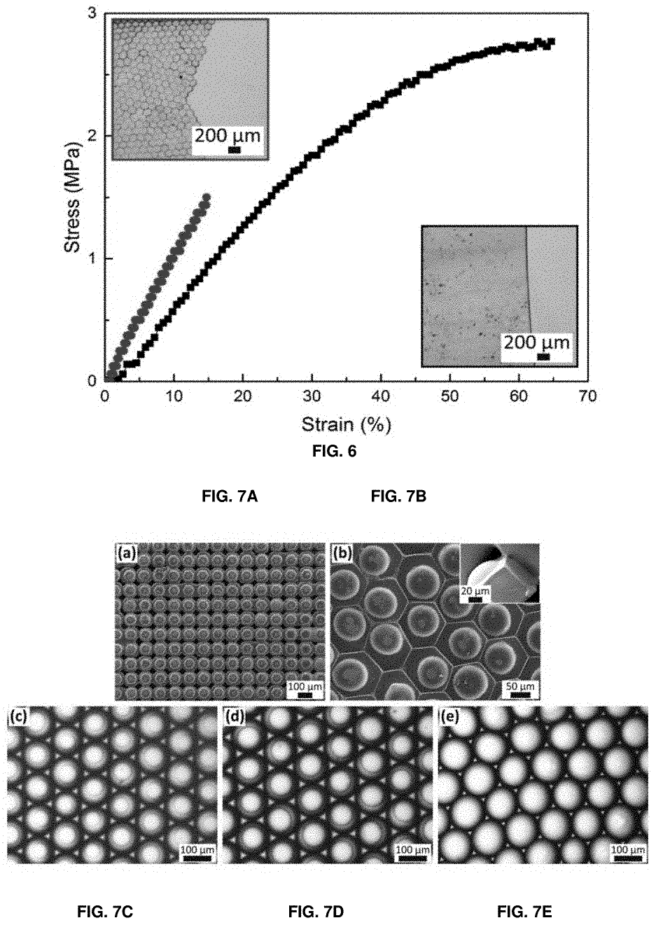

The Young's modulus of structured hydrogel sheets made of hexagonal prismatic particles is approximately 50% higher: 12.0±1.0 MPa, as shown in stress-strain curve in FIG. 6 and summarized Table 1. These results indicate that the structure of the hydrogel sheets affects their mechanical properties. Moreover, they also indicate that adjacent particles are covalently crosslinked. However, the fracture strength of structured sheets is much lower than that of unstructured counterparts, as shown in FIG. 6. Structured sheets rupture along the boundaries of particles, as shown in the optical micrograph in the inset of FIG. 6. These results indicate that the crosslink density between adjacent particles is significantly lower than that within the hydrogel particles. Moreover, the crosslink density between adjacent particles must be significantly lower than that in the corresponding unstructured counterpart.

The increase in the Young's modulus and decrease in fracture toughness caused by the structuring of hydrogel sheets indicates that the crosslink density is heterogeneous. It is higher inside individual hydrogel particles compared to bulk samples but lower between adjacent particles. Theoretically, the crosslink density should become more homogeneous if the density of covalent crosslinks is increased. To test this expectation, the inventors produced microparticles from PEG-DA with a lower molecular weight, PEG575-DA, which results in a higher density of covalent crosslinks. Also for these samples, the Young's modulus of structured hydrogels is significantly higher than that of unstructured counterparts. However, in agreement with the expectation, the difference in Young's moduli decreased to 25% as summarized in Table 1.

The density of physical crosslinks depends on the density of polymers and hence on the initial monomer concentration. To test the influence of the physical crosslink density on the mechanical properties, the monomer concentration was reduced to 40 wt %. The reduced density of physical crosslinks results in a lower Young's modulus, as summarized in Table 1. The Young's modulus of structured sheets is again approximately 50% higher than that of unstructured counterparts. This result indicates that the crosslink density is again much higher in the interior of hydrogel particles than between adjacent ones. By further reducing the monomer concentration to 30 wt %, the crosslink density between adjacent particles is further decreased, and reaches so low values that integral films cannot form any more. Hence, there is a minimum monomer concentration below which no structured hydrogel films can be formed any more. Because the crosslink density between adjacent particles is much lower than that in the interior of particles, the minimum monomer concentration required to form structured hydrogel sheets is significantly above that required to convert drops into hydrogel microparticles.

Drops spontaneously assemble into the thermodynamically most favorable hexagonal close packed structure, if deposited on a flat substrate, resulting in hexagonal prismatic particles. To test if the drop arrangement can be controlled and therefore the structure of the resulting films, drops were deposited on a PDMS substrate containing 95 μm diameter hexagonal wells. The wells are arranged in a cubic lattice with a distance of 130 μm, which is slightly larger than the drop diameter. After drops are illuminated with UV light, a connected sheet made of spherical particles that are arranged in a cubic lattice can be easily detached. The bottom surfaces of these particles are hexagonal due to the hexagonal wells used to arrange the drops, as shown in the SEM image in FIG. 7A. These results indicate that the shape and morphology of hydrogel sheets can be tuned with templates. However, it is much easier to produce structured sheets without the need of templates.

To test if the roughness of hydrogel sheets formed from self-assembled drops can be tuned, the inventors modified the polymerization kinetics of the hydrogel particles. The density of hydrated hydrogel particles is approximately 10% below that of a monomer-containing solution. Hence, if not all the monomers are crosslinked, hydrogel microparticles should rise to the top of the emulsion drops. If the remaining monomers are crosslinked only after the hydrogel microparticle creamed, the resulting particles should contain a semi-spherical surface. To test this expectation, the inventors polymerized the particles in two steps: at the beginning, the drops were illuminated with UV light for 3 seconds immediately after they have been assembled into the hexagonal structure, when they are still spherical. While the majority of the oil evaporates, the drops deform into hexagonal prisms. During this time, the small hydrogel microparticles that formed inside the drops by consuming parts of the monomers rise to their surface. When all the drops deformed into hexagonal prisms, the inventors exposed them again to UV light for 15 s. Thereby, sheets composed of hexagonal prisms were obtained, where each prism contains a semi-spherical dome, as shown in the SEM image in FIG. 7B. The size of the dome can be tuned with the distance between the UV light source and the sample, as shown in the optical microscopy images in FIGS. 7C-7E. In FIGS. 7C-7E the distance was 5cm. Hence, micrometer-scale surface roughness can be introduced into the structured sheets by tuning the polymerization kinetics of the monomers.

It is possible to achieve the same effect if monomers contained in spherical drops start to polymerize and form spherical particles. If these drops self-assemble on substrates and the surrounding oil is evaporated such that the drops deform, they attain a hexagonal prismatic form. If the diameters of the polymerized spherical particles contained in the drop are larger than the height of the hexagonal prisms, domes can form in the particles.

The main advantage of using drops to build structured hydrogel sheets is the ease with which their composition can be varied locally. To exploit this feature, the inventors produced two different batches of drops with identical sizes but different compositions. Fluorescein-labeled dextran was added to one batch of PEG-DA containing drops. The other batch of PEG-DA containing drops did not encompass any fluorescent label. The two batches are mixed before drops are assembled on a PDMS substrate and UV illuminated to be converted into structured sheets. The composition of these sheets changes over very short length scales, as shown by the abrupt changes in the fluorescence intensity in FIGS. 8A-8B. To change the composition of the structured sheets more frequently, smaller drops can be formed. The resulting sheets are made of much smaller hexagonal prisms that allow abrupt changes in the hydrogel composition with a higher frequency, as shown in the optical micrograph in FIGS. 8C-8D.

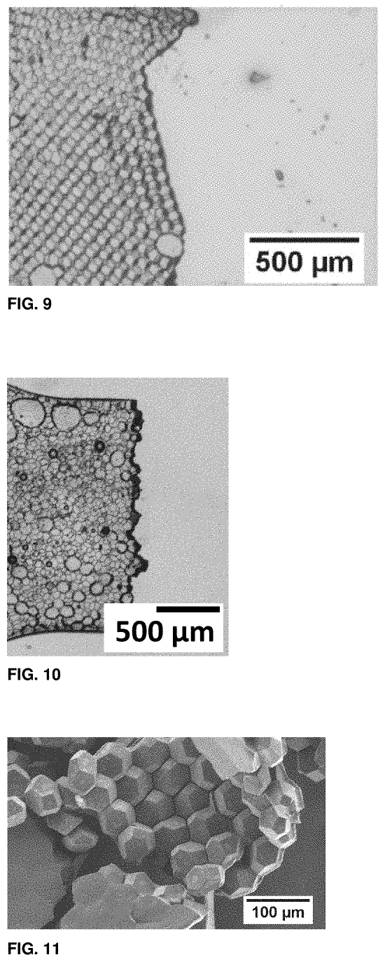

To increase the thickness and strength of the polymeric structures, two layers of drops loaded with PEG-diacrylate monomers with a molecular weight of 700 Da and a photoinitiator, 2-Hydroxy-2-methylpropiophenone, were deposited on a substrate. The surrounding oil was evaporated such that the drops deformed into hexagonal prisms before the polymerization reaction of the monomers was initiated through exposure to UV illumination. The resulting granular hydrogels, as shown in the optical micrograph in FIG. 9, composed of a bilayer of hexagonal prismatic cells are significantly stiffer than hydrogels composed of a monolayer of hexagonal prismatic cells. The bilayer exhibited a Young's modulus of 24-30 MPa.

Granular hydrogels composed of cells that are covalently crosslinked to each other break along the grain boundaries. To increase the break path, cracks must propagate before the films break. To improve resistance to breakage, polymeric structures using polydisperse drops that are loaded with PEG-diacrylate monomers and a photoinitiator were produced. These drops were assembled on a substrate, the surrounding oil was evaporated such that the drops deformed before the monomers contained in the drops were polymerized through exposure to UV light. The resulting granular hydrogels, as shown in the optical micrograph in FIG. 10, are tougher than counterparts with crystalline structures. As such, using polydisperse cells (characterised as particles of different sizes) instead of monodisperse structures (where the cells are all identically sized) can reduce or minimise structure breakage.

To increase the thickness of polymeric structures, multilayers of drops loaded with PEG-diacrylate monomers with a molecular weight of 700 Da and a photoinitiator, 2-Hydroxy-2-methylpropiophenone, were deposited on a substrate. The surrounding oil, HFE7500 or HFE7100) was allowed to evaporate such that the drops deformed before the structure was exposed to UV light to initiate the polymerization reaction. Granular hydrogels composed of polygonal cells were obtained and are shown in the scanning electron micrograph of FIG. 11.

Polymeric structures of up to ten layers were prepared but this does not represent an upper limit on the potential layers possible. This is especially true when thermal polymerization initiators are used.

### REFERENCES

P. Nicolson and J. Vogt, Biomaterials, 2001, 22, 3273-3283;

Y. Qiu and K. Park, Advanced Drug Delivery Reviews, 2001, 53, 321-339;

H. Shewan and J. Stokes, Journal of Food Engineering, 2013, 119, 781-792;

J. Burdick and W. Murphy, Nature Communications, 2012, 3, 1269;

K. Lee and D. Mooney, Chemical Reviews, 2001, 101, 1869-1880;

M. Lutolf and J. Hubbell, Nature Biotechnology, 2005, 23, 47-55;

X. Chen, A. Dunn, G. Sawyer, and M. Sarntinoranont, Journal of biomechanical engineering, 2007, 129, 156-163;

D.-H. Kim and D. Martin, Biomaterials, 2006, 27, 3031-3037

J.-T. Zhang, T. Keller, R. Bhat, B. Garipcan, and K. Jandt, Acta biomaterialia, 2010, 6, 3890-3898;

B. Trappmann, J. Gautrot, J. Connelly, D. Strange, Y. Li, M. Oyen, M. Stuart, H. Boehm, B. Li, V. Vogel, J. Spatz, F. Watt, and W. Huck, Nature materials, 2012, 11, 642-9;

K. Saha, A. Keung, E. Irwin, Y. Li, L. Little, D. Schaffer, and K. Healy, Biophysical journal, 2008, 95, 4426-38;

S. Peyton, C. Raub, V. Keschrumrus, and A. Putnam, Biomaterials, 2006, 27, 4881-93;

Anseth, Bowman, and Brannon-Peppas, Biomaterials, 1996, 17, 1647-57;

Q. Li, D. Barrett, P. Messersmith, and N. Holten-Andersen, ACS nano, 2016, 10, 1317-24;

S. Grindy, R. Learsch, D. Mozhdehi, J. Cheng, D. Barrett, Z. Guan, P. Messersmith, and N. Holten-Andersen, Nature Materials, 2015, 14, 1210-1216;

J.-Y. Sun, X. Zhao, W. Illeperuma, O. Chaudhuri, K. Oh, D. Mooney, J. Vlassak, and Z. Suo, Nature, 2012, 489, 133-136;

S. Bryant, T. Chowdhury, D. Lee, D. Bader, and K. Anseth, Annals of biomedical engineering, 2004, 32, 407-17;

Geest, J. Urbanski, T. Thorsen, J. Demeester, and S. Smedt, Langmuir, 2005, 21, 10275-10279;

M. Goldberg, R. Langer, and X. Jia, Journal of biomaterials science. Polymer edition, 2007, 18, 241-68;

S. Sugiura, T. Oda, Y. Izumida, Y. Aoyagi, M. Satake, A. Ochiai, N. Ohkohchi, and M. Nakajima, Biomaterials, 2005, 26, 3327-31;

W. Jeong, J. Kim, S. Kim, S. Lee, G. Mensing, and D. Beebe, Lab on a chip, 2004, 4, 576-80;

K. Liu, H.-J. Ding, J. Liu, Y. Chen, and X.-Z. Zhao, Langmuir, 2006, 22, 9453-9457;

A. Sawhney, C. Pathak, and J. Hubbell, Macromolecules, 1993, 26, 581-587;

R. Yoshida, K. Uchida, Y. Kaneko, K. Sakai, A. Kikuchi, Y. Sakurai, and T. Okano, Nature, 1995, 374, 240-242;

B. Lee, J. Dalsin, and P. Messersmith, Biomacromolecules, 2002, 3, 1038-47;

N. Holten-Andersen, M. J. Harrington, H. Birkedal, B.P. Lee, P. B. Messersmith, K. Y. C. Lee, and J. H. Waite, Proceedings of the National Academy of Sciences, 2011, 108, 2651-2655;

M. Ehrbar, S. Rizzi, R. Hlushchuk, V. Djonov, A. Zisch, J. Hubbell, F. Weber, and M. Lutolf, Biomaterials, 2007, 28, 3856-3866;

J. Burdick and W. Murphy, Nature Communications, 2012, 3, 1269;

D. Barrett, D. Fullenkamp, L. He, N. Holten-Andersen, K. Lee, and P. Messersmith, Advanced Functional Materials, 2013, 23, 1111-1119;

Q. Wang, J. Mynar, M. Yoshida, E. Lee, M. Lee, K. Okuro, K. Kinbara, and T. Aida, Nature, 2010, 463, 339-343;

J. Yeh, Y. Ling, J. Karp, J. Gantz, A. Chandawarkar, G. Eng, J. Blumling, R. Langer, and A. Khademhosseini, Biomaterials, 2006, 27, 5391-8;

J. Rolland, B. Maynor, L. Euliss, A. Exner, G. Denison, and J. DeSimone, Journal of the American Chemical Society, 2005, 127, 10096-10100;

A. Revzin, R. Russell, V. Yadavalli, W.-G. Koh, C. Deister, D. Hile, M. Mellott, and M. Pishko, Langmuir, 2001, 17, 5440-5447;

M. Mannoor, Z. Jiang, T. James, Y. Kong, K. Malatesta, W. Soboyejo, N. Verma, D. Gracias, and M. McAlpine, Nano Letters, 2013, 13, 2634-2639;

L. A. Hockaday, K. H. Kang, N. W. Colangelo, Y. C. P. Cheung, B. Duan, E. Malone, J. Wu, L. N. Girardi, L. J. Bonassar, H. Lipson, C.C. Chu, and J. T. Butcher, Biofabrication, 2012, 4, 035005;

C. Smith, A. Stone, R. Parkhill, R. Stewart, M. Simpkins, A. Kachurin, W. Warren, and S. Williams, Tissue engineering, 2004, 10, 1566-76;

S. Murphy and A. Atala, Nature Biotechnology, 2014, 32, 773-785;

N. Vogel, M. Retsch, C.-A. Fustin, A. del Campo, and U. Jonas, Chemical Reviews, 2015, 115, 6265-6311;

A. Lyon, J. Debord, S. Debord, C. Jones, J. McGrath, and M. Serpe, The Journal of Physical Chemistry B, 2004, 108, 19099-19108;

J. Debord and A. Lyon, The Journal of Physical Chemistry B, 2000, 104, 6327-6331;

S. Schmidt, T. Hellweg, and R. von Klitzing, Langmuir, 2008, 24, 12595-12602;

G. Zhang, D. Wang, Z.-Z. Gu, J. Hartmann, and H. Mohwald, Chemistry of Materials, 2005, 17, 5268-5274;

D. Suzuki, J. McGrath, H. Kawaguchi, and A. Lyon, The Journal of Physical Chemistry C, 2007, 111, 5667-5672;

M. Kumoda, M. Watanabe, and Y. Takeoka, Langmuir, 2006, 22, 4403-4407;

S. Köster, F. Angile, H. Duan, J. Agresti, A. Wintner, C. Schmitz, A. Rowat, C. Merten, D. Pisignano, and A. Griffiths, Lab on a Chip, 2008, 8, 1110-1115;

T. Rossow, J. Heyman, A. Ehrlicher, A. Langhoff, D. Weitz, R. Haag, and S. Seiffert, Journal of the American Chemical Society, 2012, 134, 4983-4989;

S. Allazetta and M. Lutolf, Current opinion in biotechnology, 2015, 35, 86-93.

1. A polymeric structure, comprising a plurality of spherical or polyhedral polymeric cells bound through their sidewalls via a connection element, wherein the polymeric cells and the connection element have a different Young's modulus and/or the polymeric cells and the connection element have different crosslink densities.

2. (canceled)

3. The polymeric structure of claim 1, wherein the polymeric cells and the connection element are composed of the same polymeric matrix.

4. The polymeric structure of claim 1, wherein the Young's modulus of the polymeric cells is higher compared to the Young's modulus of the connection element; preferably, wherein the difference in Young's modulus of the polymeric cells and connection element is about 1 kPa to about 100 MPa, about 10 kPa to about 100 MPa, about 100 kPa to about 100 MPa, about lkPa to about 10 MPa, about 10 kPa to about 10 MPa, about 100 kPa to about 10 MPa, about 1 kPa to about 1000 kPa, about 1 kPa to about 100 kPa or about 1 kPa to about 10 kPa.

5. (canceled)

6. The polymeric structure of claim 1, wherein the crosslink density of the polymeric cells is higher compared to the crosslink density of the connection element.

7. The polymeric structure claim 1, wherein the cros slink density of the polymeric cells is lower compared to the cros slink density of the connection element.

8. The polymeric structure of claim 1, wherein the cells are bound through their sidewalls chemically, ionically or via physical crosslinks.

9. The polymeric structure of claim 1, wherein at least part of the plurality of polymeric cells are adjacently disposed in a crystalline lattice along or within the structure, preferably, wherein it is shaped as a flat sheet or a three-dimensional solid, and more preferably, wherein it is a flat sheet comprising a non-flat surface.

10-11. (canceled)

12. The polymeric structure of claim 1, wherein at least part of the plurality of polymeric cells are labelled with a marker or a dye, such as a fluorescent dye, crystal label or electronic marker.

13. A method for producing a polymeric structure comprising a plurality of spherical or polyhedral polymeric cells bound through their sidewalls via a connection element, wherein the polymeric cells and the connection element have a different Young's modulus, said method comprising the steps of:
providing a plurality of spherical or polyhedral polymeric cells disposed on a support substrate;
providing a connection element between the sidewalls of the polymeric cells; and
allowing a crosslinking reaction between the polymeric cells and the connection element by providing a crosslinking trigger; and/or
a method for producing a polymeric structure comprising a plurality of spherical or polyhedral polymeric cells bound through their sidewalls via a connection element, wherein the polymeric cells and the connection element have different crosslink densities, said method comprising the steps of:
providing a plurality of spherical or polyhedral polymeric cells disposed on a support substrate;
providing a connection element between the sidewalls of the polymeric cells; and
allowing a crosslinking reaction between the polymeric cells and the connection element by providing a crosslinking trigger.

14. (canceled)

15. The method of claim 13, wherein the step of providing a plurality of spherical or polyhedral polymeric cells disposed on support substrate in a packed arrangement comprises:
providing a plurality of drops comprising chemical precursors of a polymeric matrix composing the polymeric cells, said drops being arranged into a non-chemically bound packed arrangement on said support substrate; and
starting a solidification reaction of the polymeric matrix composing the polymeric cells.

16. The method of claim 15 wherein the chemical precursors comprise a minimum monomeric concentration of greater than 30 wt %, greater than 40 wt %, greater than 50 wt %, or greater than 60 wt %.

17. The method of claim 13, comprising the steps of:
a) providing an emulsion comprising:
a first phase comprising a plurality of drops of chemical precursors of a polymeric matrix dispersed into an immiscible second phase; and
a crosslinker;
b) disposing said emulsion on a support substrate so that the drops assemble into a non-chemically bound packed structure;
c) starting a solidification reaction of the polymeric matrix by providing a crosslinking trigger; and
d) during the solidification reaction, and before the solidification reaction is complete, allowing the removal of the second phase.

18. The method of claim 17, wherein the first phase is an aqueous phase and the second phase is an organic phase.

19. The method of claim 17, wherein step a) of providing an emulsion is performed through a microfluidic device.

20. The method of claim 13, wherein surface modifying agent(s) is/are used to form the connection element.

21. The method of claim 13, wherein the step of providing a connection element between the sidewalls of the polymeric cells comprises:
disposing a liquid or semi-solid connection element between the plurality of polymeric cells; and
optionally allowing solidification of said liquid or semi-solid connection element.

22. The method of claim 13, wherein the solidification is caused by polymerisation, gelation, or solvent removal, preferably polymerisation.

23. The method of claim 13, wherein the crosslinking trigger is a thermal trigger, a light trigger, a chemical trigger such as a complexation agent, or a catalyst, preferably a thermal trigger or a light trigger, preferably, wherein the crosslinking trigger is UV light.

24. (canceled)

25. The method of claim 13, further comprising a step of labelling at least part of the plurality of polymeric cells or drops with a marker or a dye, such as a fluorescent dye, crystal label or electronic marker.

26. A pharmaceutical composition or a scaffold for tissue engineering comprising the polymeric structure of claims 1, or polymeric structures made according to the process of claims 13; or a physical, non-printed polymeric data matrix tag, wherein the physical, non-printed polymeric data matrix tag comprises a polymeric structure according to claim 9 or claim 10 in which part of the plurality of the polymeric cells are labelled with a marker or a dye, such as a fluorescent dye and/or a dye that is visible to the human eye.

27-28. (canceled)
